(19) United States
(12) Patent Application Publication
(10) Pub. No.: US 2025/0256053 A1
(43) Pub. Date: Aug. 14, 2025

(54) Cognition and memory enhancement via multiple odorant stimulation based on a profile of a user's scent experiences

(71) Applicant: Science Lab 3 Inc., Dana Point, CA (US)

(72) Inventor: Young Tang, Downey, CA (US)

(73) Assignee: Science Lab 3 Inc., Dana Point, CA (US)

(21) Appl. No.: 18/939,384

(22) Filed: Nov. 6, 2024

Related U.S. Application Data
(60) Provisional application No. 63/559,447, filed on Feb. 29, 2024.
(60) Provisional application No. 63/553,000, filed on Feb. 13, 2024.

(51) Int. Cl. A61M 21/00

(52) U.S. Cl. CPC A61M 21/00; A61M 2021/0016; A61M 2205/07; A61M 2205/3303; A61M 2205/52

(57) ABSTRACT

Disclosed herein are methods, kits, and devices for improving cognitive function and memory through olfactory stimulation. In some embodiments, olfactory stimulation is performed by releasing one or more scents according to an olfactory stimulation regimen or schedule. The scents can include scents that are familiar to a patient as derived from a patient profile. The methods, kits and devices described herein can provide a large impact on cognition with minimal effort and cost. They can be used widely and effectively among older adults, children, and other populations in need of improved cognitive performance.

This application claims the benefit of U.S. Provisional Application No. 63/559,447, filed Feb. 29, 2024 and U.S. Provisional Application No. 63/553,000, filed Feb. 13, 2024, both of which are incorporated herein by reference in their entireties.

### BACKGROUND OF THE INVENTION

### Field of the Invention

The invention described herein relates to therapeutic systems and methods operating via olfaction.

### Background Art

Environmental enrichment has been shown to have a positive impact on cognitive function such as in ameliorating cognitive decline in various animal models. Enriching an environment can be accomplished in a variety of ways including, but not limited to, the introduction of various objects, sounds, smells, colors, animals, etc. Thousands of research papers reaching this conclusion have been published and the cognitive benefits have been shown to reduce or overcome animal models of human neurological disorders such as Alzheimer's disease, memory loss, vascular dementia, neuronal death in aging, traumatic brain injury, head injury, Parkinson's disease, seizures, stroke, multiple sclerosis, anxiety, autism, ADHD, Huntington's disease, Down Syndrome, stress, depression, cerebral palsy, chemo-brain, schizophrenia, prenatal alcohol syndrome, lead exposure, addiction and cancer, to name a few. Noticeable behavioral changes have been observed in children with autism after as little as six months of environmental enrichment exercises.

Of the various stimulants used in environmental enrichments, it has been found that cognition is strongly associated with olfaction. Olfaction is the only sense that has a large, direct pathway to the cognitive and emotional areas of the brain. Loss of olfaction precedes the memory loss from aging and from all forms of dementia. Olfactory loss triggers a massive loss of neurons in the brain. After the age of 50, it has been found that olfactory ability accurately predicts all-cause mortality within the next five years.

Biologically, olfactory stimulation activates the entorhinal cortex. The entorhinal cortex diminishes with age and other factors such as Alzheimer's and other forms of dementia. As the entorhinal cortex diminishes, it releases the dentate gyrus and CA3 (hippocampal subfields) from inhibition, thereby interfering with memory. Thus, a restoration of olfaction should increase neurogenesis and/or neural complexity in the entorhinal cortex, thereby normalizing dentate/CA3 activity and restoring memory.

Interventions based on environmental enrichment can be expensive or difficult to maintain, as in the case of exercise. Currently, there is a lack of environmental enrichment modalities that effectively improve cognitive function and memory while being low cost, and easy to use or maintain. More specifically, there is a lack of environmental enrichment modalities that exploits the strong association that olfaction has with the cognitive and emotional areas of the brain.

### BRIEF DESCRIPTION OF THE DRAWINGS/FIGURES

Features and advantages of embodiments of the invention will be apparent and elucidated from the following description of embodiments of the present invention, reference being made to the accompanying drawings.

FIG. 1 is a graph showing a decline in cognitive functions over time;

FIG. 2 is a timeline depicting the administration of an embodiment of a method of the invention;

FIG. 3 is a timeline depicting an embodiment of a treatment schedule of a method of the invention;

FIG. 4 is a graph showing a human sleep cycle;

FIG. 5 is a diagram of a fragrance wheel;

FIG. 6 is a chart showing an embodiment of a scent rotation of the invention;

FIG. 7 is a diagram showing Henning's Prism;

FIG. 8 is a diagram of a fragrance wheel formed from one side of Henning's Prism;

FIG. 9 is a flow chart illustrating the incorporation of scents into a scent rotation, where at least some of the scents are familiar to a user, according to an embodiment;

FIG. 10 is a flow chart illustrating the use of a user questionnaire in identifying familiar scents, according to an embodiment;

FIG. 11 is a perspective view of an embodiment of a device of the invention;

FIG. 12 is a cutaway view of the embodiment of the device of FIG. 11;

FIG. 13 is a perspective view of an embodiment of a cartridge of the invention;

FIG. 14 is a cutaway view of the cartridge of FIG. 13;

FIG. 15 is an elevation of an embodiment of a scent pad package of the invention;

FIG. 16 is a perspective view of an embodiment of a gel tab of the invention;

FIG. 17 is a side elevation of an embodiment of a CPAP mask of the invention;

FIG. 18 is a graph showing a change in Stroop score using an embodiment of the invention;

FIG. 19 is a graph showing a change in Rey Auditory Verbal Learning Test score using an embodiment of the invention;

FIG. 20 is a graph showing a change in Wechsler Adult Intelligence Scale-III-Letter-Number Sequencing score using an embodiment of the invention; and,

FIG. 21 is a graph showing a change in Wechsler Adult Intelligence Scale-III Backwards Digit Span score using an embodiment of the invention.

Further embodiments, features, and advantages of the present invention, as well as the operation of the various embodiments of the present invention, are described below with reference to the accompanying drawings.

### DETAILED DESCRIPTION OF THE INVENTION

A preferred embodiment of the present invention is now described with reference to the figures, where like reference numbers indicate identical or functionally similar elements. Also in the figures, the leftmost digit of each reference number corresponds to the figure in which the reference number is first used. While specific configurations and arrangements are discussed, it should be understood that this is done for illustrative purposes only. A person skilled in the relevant art will recognize that other configurations and arrangements can be used without departing from the spirit and scope of the invention. It will be apparent to a person skilled in the relevant art that this invention can also be employed in a variety of other systems and applications.

### Definitions

All scientific and technical terms used in this application have meanings commonly used in the art unless otherwise specified. As used in this application, the following words or phrases have the meanings specified.

As used herein, “releasing” a scent to a subject, or “exposing” a subject to a scent, means that the scent is presented into the environment of the subject in a manner sufficient for stimulation of the subject's olfactory system.

As used herein, “decreases” or “increases” means reduces or raises, respectively, by a detectable or noticeable amount. In some embodiments, such a decrease or increase is measured using one of the assessment tools described herein. In some embodiments, the decrease or increase represents a “significant difference”.

As used herein, “improves” means results in an improved state, for example, the amelioration of symptoms of an adverse condition, or the enhancement of a desired condition, such as memory.

As used herein, a “significant difference” or “significantly different” means a difference that can be detected in a manner that is considered reliable by one skilled in the art, such as a statistically significant difference, or a difference that is of sufficient magnitude that, under the circumstances, can be detected with a reasonable level of reliability. In one example, an increase or decrease of 10% relative to a reference value is a significant difference. In other examples, an increase or decrease of 20%, 30%, 40%, or 50% relative to the reference value is considered a significant difference. In yet another example, an increase of two-fold relative to a reference value is considered significant. The reference value can be, for example, an untreated subject, or a baseline (pretreatment) value for the same subject.

As used herein, ranges and amounts can be expressed as “about” a particular value or range. About also includes the exact amount. Hence “about 5 minutes” means “about 5 minutes” and also “5 minutes.” Generally, the term “about” includes an amount that is from 10% below to 10% above the particular value or range. For example, “about 10 minutes” means “from 9 minutes to 11 minutes.”

### Environmental Enrichment

In more than a dozen animal models of human neurological conditions, environmental enrichment has been shown to ameliorate their human-like symptoms, including the cognitive decline in aging (Patel, et al., 2012; Segovia, et al., 2006; Valero, et al., 2007) and in Alzheimer's disease (Arendash, et al., 2004; Arranz, et al., 2011; Basak, et al., 2008; Berardi, et al., 2007; Costa, et al., 2007; Jankowsky, et al., 2015; Lazarov, et al., 2005; Polito, et al., 2014). In humans, environmental enrichment, including exercise, computer games, social behavior and nutrition have been shown to reduce the risk of cognitive decline in aging and disease (Herzog, et al., 2008; Kirk-Sanchez and McGough, 2014; Klimova, 2016; Williams and Kemper, 2010), but it has been difficult to show that cognitive decline can be reversed, and when there are such improvements, the effects tend to be modest in size (Herzog, et al., 2008; Rodakowski, et al., 2015). Moreover, it has been shown to be difficult to keep people on such interventions as a routine program of physical exercise (Williams, et al., 2007).

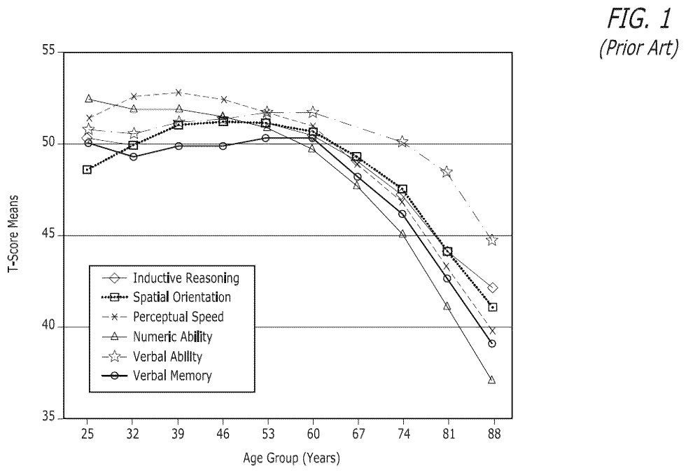

The olfactory system experiences a clear deterioration in older adults, with 18% of older adults having olfactory impairment and 46% of those over 80 years old having very limited olfactory ability (Doty, et al., 1984; Hoffman, et al., 1998; Liu, et al., 2016; Murphy, et al., 2002; Pinto, et al., 2015; Toussaint, et al., 2015). The initiation of this decline parallels that seen for cognitive ability in older adults (Park, et al., 2003; Salthouse, 2009). The decline in cognitive function with age is illustrated in FIG. 1, which shows an initial decline around the age of 60 that continues as the individual ages with inductive reasoning, spatial orientation, perceptual speed, numeric ability, verbal ability, and verbal memory all being affected. The olfactory system has direct projections to cognitive areas, unlike other sensory systems, and the loss or compromise of the olfaction system results in massive volume loss in these cognitive areas in humans at any age (Bitter, et al., 2010a; 2010b; 2011; Yao, et al., 2014). Normal human aging is accompanied by a deterioration of olfactory abilities (Dong, et al., 2017; Hoffman, et al., 2016; Seubert, et al. 2017), along with the deterioration of olfactory projection sites, which include the cognitive areas of the brain (Kollndorfer, et al., 2015; Segura, et al., 2013). Moreover, a degradation of olfactory ability predicts both an elevated risk of minimal cognitive impairment (MCI) and which of those with MCI will go on the develop Alzheimer's disease (Adams, et al., 2017; Devanand, et al., 2000; Lafaille-Magnan, et al., 2017; Peter, et al., 2003; Roberts, et al., 2016; Schubert, et al., 2008; 2017; Swan and Carmelli, 2002). Remarkably, olfactory function also predicts all-cause mortality in older adults (Gopinath, et al., 2011; Pinto, et al., 2014).

The loss of olfactory function precedes or parallels the initiation of a variety of cognitive disorders such as Alzheimer's disease, Parkinson's disease, Lewy body dementia, fronterotemporal dementia, Creutzfeldt-Jakob disease, minimal cognitive impairment, and schizophrenia (Conti, et al., 2013; Devanand, et al., 2000; Devanand, et al., 2010; Doty, et al., 1988; Li, et al., 2010; Luzzi, et al., 2007; Meusel, et al., 2010; Nguyen, et al., 2010; Parrao, et al., 2012; Ponson, et al., 2004; Ross, et al., 2006; Tabaton, et al., 2004; Wattendorf, et al., 2009). Given that these cognitive disorders have widely differing etiologies, it raises the possibility that the loss of olfactory stimulation contributes to the decline in cognitive ability in each of these disorders.

In humans who have experienced olfactory loss due to a variety of problems, such as post-infectious olfactory dysfunction, head trauma, Parkinson's, and aging) increased olfactory experiences has been shown to improve olfactory identification, olfactory discrimination, and to lesser extent, olfactory threshold (Damm, et al., 2014; Geißler, et al., 2014; Haehner, et al., 2013; Hummel, et al., 2009; Konstantinidis, et al., 2013; Patel, et al., 2017). These results were achieved through exposure to four odorants taken from each of four odor groups: resinous (eucalyptus), flowery (rose), fruity (lemon), and aromatic (clove). There are further improvements in olfactory ability with increased duration of exposure, increased concentration of the odorants and an increased number of odorants (Altundag, et al., 2015; Damm, et al., 2014; Konstantinidis, et al., 2016). In addition to improvements in sensory ability, older adults exposed to increased olfactory stimulation have an improvement in their cognitive function, as evidenced by increased verbal fluency, an improvement in their depressive symptoms, and an improved sense of wellbeing (Wegener, et al., 2018). Olfactory stimulation in older adults also decreases falls in this group (Sakamoto, et al., 2012).

Individuals with autism have olfactory dysfunction, both with their abnormal sniff response to odors of different valences (Rozenkrantz, et al., 2015) and to social odors (Endevelt-Shapira, et al. 2018). They also have abnormal olfactory responses (Boudjarane, et al., 2017; Tonacci, et al., 2017). Diverse rodent models of autism have their human-like symptoms of this disorder ameliorated when they are placed into an enriched environment (Kerr, et al., 2010; Kondo et al., 2008; Lacaria, et al., 2012; Lonetti et al., 2010; Nag et al., 2009; Restivo et al., 2005; Reynolds, et al., 2013; Schneider & Przewlocki, 2005; Schneider et al., 2006).

Environmental enrichment, which included olfactory stimulation that was paired with tactile stimulation has been successfully used to treat children with classic autism (Woo and Leon, 2013, Woo, et al., 2015). In two randomized clinical trials, parents delivered novel multisensory stimulation, featuring olfactory stimulation, each morning and evening for 15-30 min over the course of six months. 42% of the enriched children had a major improvement in their symptoms as measured on the subjective Childhood Autism Rating Scale, compared to 7% of the standard-care controls who had that kind of improvement. IQ increased by more than 8 points for enriched children, compared to about a point of improvement in controls. The Short Sensory Profile revealed an 11-point improvement for enriched children and about a point for controls. Receptive language, as measured by the objective Reynell Developmental Language Scales, improved by more than 200% in enriched with autism and less than 20% for controls. Finally, after 6 months, 21% of the children with autism were considered to have lost that diagnosis using the objective Autism Diagnostic Observation Schedule and no control child reached that level of improvement.

A review of the outcomes of over 1,000 children along the entire autism spectrum who were being given this treatment at home (Aronoff, et al., 2016) revealed that these children did better than those in the university clinical trials, with an effect size of 1.85. Not only did the core symptoms of autism improve, but the co-morbid symptoms, which accompany virtually every child with this disorder have, including: sensory processing, self-awareness, communication, mood, sleeping, eating, motor skills, learning, memory, anxiety and attention span. This treatment had similar effects on children across the entire autism spectrum, both girls and boys improved equally, and the treatment worked for all ages tested (2-18 years old). There was also a dose/response outcome for parental compliance and the symptom improvement of their children.

### Schedules

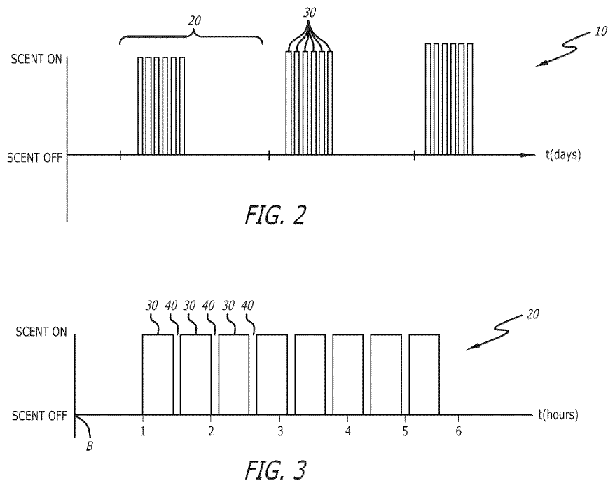

Referring now to the figures, and first to FIGS. 2 and 3, there is shown a general illustration of a method 10 of the invention. The method 10 generally involves subjecting a treatment user to a treatment schedule 20 of scent intervals 30 separated by breaks 40. The treatment schedule can be administered for several hours, days, weeks, months or years, depending on the treatment user and the desired results. The treatment schedule can be repeated on a daily, weekly, or monthly basis. As there are no negative side-effects or downsides to the treatment method 10, other than perhaps the expense of the fragrances, benefit may be found in continuing the schedule 20 indefinitely.

FIG. 3 provides a non-limiting example of a treatment schedule 20 that is initiated upon bedtime B. Bedtime is selected due to the ease with which the treatment may be delivered to a person not moving around, and the effectiveness which a scent may be delivered to a person in a relatively-small, enclosed room, such as a bedroom. It is noted however that treatment effectiveness may not be diminished if the treatment is delivered during the day, such as to a person sitting at a computer, driving a vehicle, or is otherwise stationary relative to the delivery device; or if the delivery device moves with the user throughout the user's daily activities, such as would be the case with a wearable device.

In the embodiment of FIG. 3, the user activates a scent-delivery device (detailed below) that can include a timer that is set to deliver various odors at predetermined intervals. In this example, the intervals 30 are approximately 30 minutes long and are separated by breaks 40 that are 5 minutes in duration. It is to be understood, however, that these interval times and break times are merely non-limiting examples and may vary based on user preference, subjective results, doctor recommendations based on empirical evidence, sleep duration, etc. Good results have been attained with 30-minute intervals with 5 minute breaks, as a starting point.

The treatment schedule 20 can begin each day at a start event. For users with a regular daily routine, the start event may be a set time of day, rather than being initiated at bedtime. For example, in this embodiment, the first interval 30 of the treatment schedule 20 may begin at midnight and continue until 12:30 am. The first break 40 thus spans from 12:30 to 12:35 am and the second interval 30 begins at 12:35 am and lasts until 1:05 am. These intervals 30 and breaks repeat this pattern until a predetermined number N of intervals 30 and breaks 40 have occurred. The number N, like the interval 30 length, may be varied based on the length of sleep the user typically gets, as well as subjective or objective results. Generally, N should be at least 3 for optimal results.

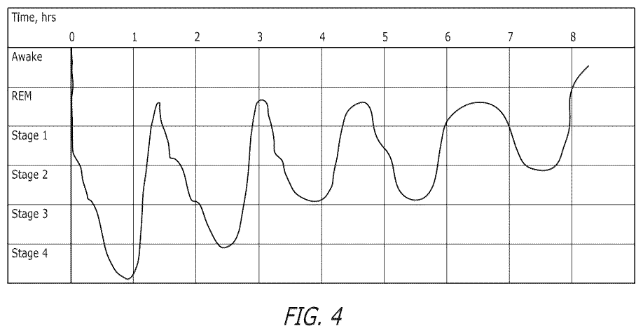

In another embodiment, the start event may be tied to the sleep cycle of the user. FIG. 4 is a diagram of the human sleep cycle 50. The sleep cycle 50 consists of various stages that repeat every 90 to 110 minutes. One model breaks the sleep cycle into five stages: Stage 1 is light sleep characterized by a decreasing amount of muscle, brain and eye activity; Stage 2 involves a slowing of heart rate and breathing pattern, as well as a slight decrease in body temperature. Stage 3 is where deep sleep begins and involves very slow delta waves produced by the brain; Stage 4 is very deep sleep with rhythmic breathing, limited muscle activity, and continued delta wave production; and the fifth stage is called the REM (Rapid Eye Movement) stage. This is the dream stage characterized by a rise in blood pressure, heart rate, breathing rate, and rapid eye movement.

Referring again to FIG. 4, it is shown that the sleep cycle begins in stage 1 and quickly progresses to stage 4 within the first hour of falling asleep. After an hour or so, the heart rate, blood pressure, and breathing rate increase rapidly as the person enters REM sleep, which typically lasts only about 5 minutes. After REM sleep, the cycle repeats to a certain extent.

Notably, throughout the night, the degree to which the sleeper enters deep sleep diminishes. For example, as seen in the graph of FIG. 4, during the second cycle, stage 4 is barely attained. The end of the second cycle is also marked by a REM stage that lasts 10 minutes, rather than 5 minutes. The third cycle only drops into stage 2 sleep, followed by a 15 minute REM stage. The fourth cycle also only drops into stage 2 sleep and is followed by a REM stage that lasts 30-60 minutes.

Much is still to be learned about the human sleep cycle. Scientists have often thought that experiences and lessons are converted into long term memory during sleep, and further that REM sleep plays an essential role in the acquisition of learned material, both declarative and procedural memory, both forms of Near Transfer. Researchers recently have begun to hypothesize that deep, restorative sleep, also referred to as “slow-wave sleep (SWS)” plays a significant role in declarative memory by processing and consolidating newly acquired information.

With this in mind, one aspect of the invention includes a sleep stage tracking system, such as a heart rate monitor, respiration monitor, blood pressure monitor, brain wave sensor, or any combination thereof. The sleep stage tracking system is used in conjunction with the scent delivery schedule to ensure that break intervals (periods between the scent delivery intervals) occur during sleep stages that are less effective, and that scent intervals occur during sleep stages that are of maximum effectiveness. It is likely that the treatment efficacy of the olfactory stimulation therapy of the invention may vary depending on which sleep stage the user is in. For example, it may be determined that treatment delivered during stages 3 and 4 have no effect while treatment delivered during REM is the most effective of all the stages.

Given that there would likely be no degradation in effectiveness if a scent were to be delivered during a point in the sleep cycle that is not optimal for Far Transfer, the benefit of tracking the sleep cycle may be to simply avoid the occurrence of breaks 40 that during periods of peak Far Transfer Effects. For example, if it is determined that REM sleep is the best stage of the sleep cycle for Far Transfer Effects, it may be beneficial to avoid breaks 40 during the REM stages, especially the first two to three REM stages, which last only 5-15 minutes.

Similarly, if it is determined that Stage 4 is the most beneficial time for olfactory stimulation to be administered, it would be important to ensure that intervals 30 are scheduled during the first two sleep cycles, as the first two cycles are typically the only cycles in which Stage 4 is entered.

One embodiment of the method of the invention provides intervals 30 that coincide with the sleep cycles through the use of monitoring. Because the sleep cycles coincide with variations in biological factors such as blood pressure, heart rate, respiration rate, brain wave activity, and eye movement, one skilled in the art would realize that a number of technologies exist to monitor the various sleep stages. As such, the interval 30 durations and break 40 durations could be selected to synchronize with the sleep cycles. In this embodiment, there are as few as 3 intervals or as many as 6 or 7 intervals, depending on the length of sleep by the user.

One embodiment of this synchronized method involves recording the sleep patterns of the user over a period of days to establish a typical pattern. The delivery device is then scheduled to approximate a synchronized schedule. Under this method, the user would not have to use a monitoring device unless recalibration is desired.

Another embodiment of this synchronized method involves continuous monitoring of one or more of the above-mentioned biological factors. For example, heart rate monitors are common in many forms including, but not limited to, optical wearables, chest straps, air-bed pulse detectors, and the like. The biological factor is monitored continuously and the sleep cycle is determined based on the data collected therefrom. The device then activates and deactivates the various scents according to a schedule that is created based on the data. As such, the schedule may differ slightly from evening to evening. This method may be optimally suited to users that have a varying daily routine.

In another embodiment, if the start event occurs at bedtime, the scent presentation device can disperse one or more relaxation scents known to relieve stress, anxiety, and help the user fall asleep. Examples of relaxation-scents include lavender, chamomile, or ylang-ylang; however, this is not a limiting list and can include other calming scents as well. One or more relaxation-inducing scents can be introduced to the user prior to a treatment session, essentially integrating the soothing scents as a part of the treatment routine. The relaxation scent can be chosen to be different from those in the user's scent profile, ensuring that the same scent is not repeated throughout the entire treatment schedule.

In another embodiment, at the end of a treatment schedule or as desired, the scent presentation device can disperse one or more energizing or wake-up scents such as peppermint, citrus, and eucalyptus for example and without limitation. The wake-up scent can be chosen to be different from those in the user's scent profile, ensuring that the same scent is not repeated throughout the entire treatment schedule.

### Rotations of Scents

One aspect of the invention is that some of the scents being delivered to the users may be novel. This is not to imply that such a scent may be completely unfamiliar to the user over his or her lifetime, but simply that the scent changes each interval and is not re-introduced for a predetermined number of intervals. In one embodiment, at least seven different scents are used, and no scent is repeated until the other six scents have been used. In other embodiments, a vast number of scents are used such that it is unlikely that a scent would be used twice during an entire treatment program.

As a general guideline, it is believed that the more novel a scent is (relative to other scents being presented), the greater impact it will have. Additionally, it is believed that consecutive scents should differ as much as possible during a particular daily schedule 20. In order to achieve this, one embodiment provides a rotation that avoids placing two aromas from a given scent group sequentially adjacent to each other.

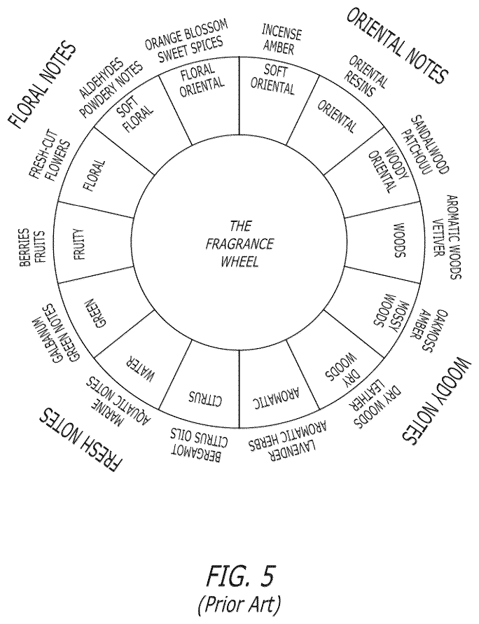

Scents are sometimes classified into scent groups, commonly referred to as the fragrance wheel or chart. There are many different fragrance charts. One widely used chart, used herein merely as an example, was developed by perfumery taxonomist Michael Edwards in 1992, and modified several times since. The 2010 version of the Michael Edwards fragrance wheel is provided as FIG. 5 and was published in 2011 in Fragrances of the World, by Michael Edwards & Co., incorporated by reference herein.

As seen in FIG. 5, there are four main fragrance families in Edwards' model: floral, oriental, woody and fresh. These are divided into subgroups and arranged such that the subgroups show relationship to each other. For example, the floral family is divided into floral, soft floral and floral oriental. The oriental family is divided into soft oriental, oriental and woody oriental. The floral oriental subgroup from the floral family is arranged next to the soft oriental subgroup from the oriental family because these two are the most similar, despite being in different families.

The fragrance wheel of FIG. 5 is useful because the further scent differences are easily visualized by their respective proximity on the wheel. For example, scents from the mossy woods subgroup are found opposite the scents from the floral subgroup. Thus, it is easy to assess that the brain will process an amber smell as very different from a fresh-cut flower smell.

In creating a rotation, a guideline for scent variety can be established using the fragrance wheel of FIG. 5. For example, a spacing of 1-5 subgroups between consecutive scents can maximize the effectiveness of the olfactory stimulation schedule 20. Similarly, it can be determined that during the course of a single daily schedule 20, at least one fragrance from each of the four families should be represented.

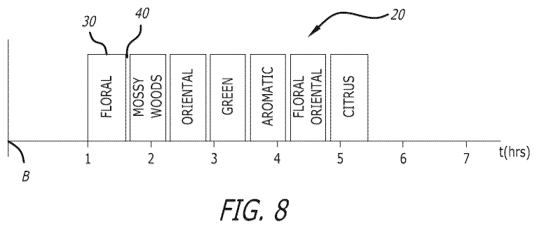

FIG. 8 provides an example rotation in which there is at least one subgroup separating consecutive intervals 30, and all four families are represented over the seven intervals 30 of the schedule 20.

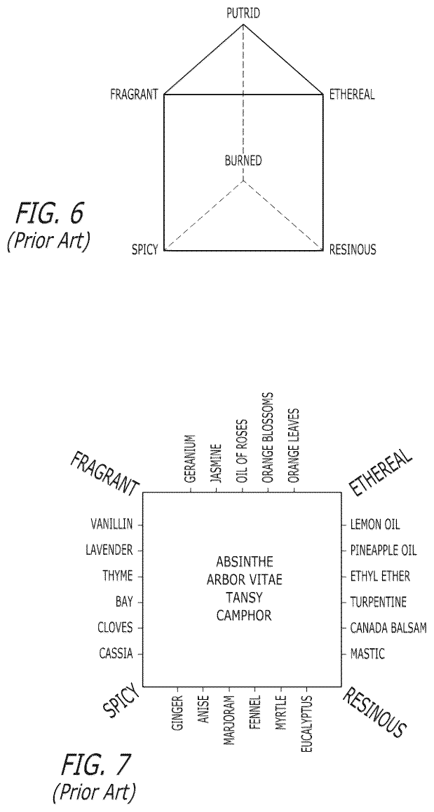

FIG. 6 provides another example of a fragrance schematic developed by Hans Henning, a German psychologist, in 1916. Henning H (1916) Der Geruch. Leipzig, Germany. The schematic of FIG. 6 is known as Henning's Prism and is a three-dimensional prism consisting of three rectangular faces, each of which could be used as a fragrance wheel. The corners of the prism include the fragrance families: fragrant (flowery or floral), ethereal (fruity), putrid, resinous, burned, and spicy. Each face shares two families with an adjoining face. Thus, one face of the prism includes the families: flowery, fruity, spicy and resinous. Another face includes the families: flowery, spicy, burnt and putrid. The third face includes the families: burnt, putrid, fruity and resinous. Because the putrid and burnt families are generally less desirable, it is thought that the face shown in FIG. 8 will be preferred. FIG. 8 takes this face and forms a wheel including the subgroups between the families.

A scent presentation device/system (discussed in greater detail below) may include a control module to disperse a first scent for a predetermined first duration, employ a break period after the predetermined first duration, and disperse a second scent for a predetermined second duration after the break period. This may be followed by subsequent scents and breaks.

The break period can be based on the type of scent just dispersed, how strong the scent is, and/or where it is located on the scent pyramid model.

The pyramid is divided into three main layers or notes, each representing a specific phase of the fragrance's life. The note of a scent may be seen as a characteristic of the scent. These notes are:

Top Notes (Head Notes): These are the initial scents that are perceived immediately upon application of a perfume, for example. Top notes are usually light, fresh, and volatile, evaporating quickly. They give the first impression of the fragrance but last for a short duration, typically ranging from a few minutes to an hour. Common top notes include citrus (like lemon and bergamot), light fruits (such as berries and grapes), and herbs (like lavender and rosemary).

Middle Notes (Heart Notes): After the top notes dissipate, the middle notes become noticeable. They are considered the heart of the fragrance, representing the main elements of the scent composition. Middle notes are more mellow and rounded than top notes and are perceived during the majority of the fragrance's wear on the skin. They typically last longer than top notes but are less volatile, usually lasting several hours. Common middle notes include floral (like rose and jasmine), spice (such as cinnamon and cardamom), and fruit scents (such as peach and plum).

Base Notes: These are the last to develop and the longest-lasting notes. Base notes give depth and solidity to the fragrance, lingering for an extended time after the top and middle notes have faded. They are usually rich, heavy, and deep scents that anchor the fragrance, preventing the lighter notes from evaporating too quickly. Common base notes include woodsy scents (like sandalwood and cedarwood), resins (such as amber and myrrh), musk, and vanilla.

In embodiments, the scent intervals, or durations, may vary according to whether a scent is a base note, a middle note, or a top note. The duration for a top note may be longer than that of a middle note. The duration for a middle note may be longer than that of a base note.

In embodiments, the break period between scent dispersions (the dispersion break period) can be longer following base notes than a break following middle notes, which in turn may be longer than a break following top notes. The break period can also be based on the room size, number of windows, available air ducts, type of scents, or a combination thereof.

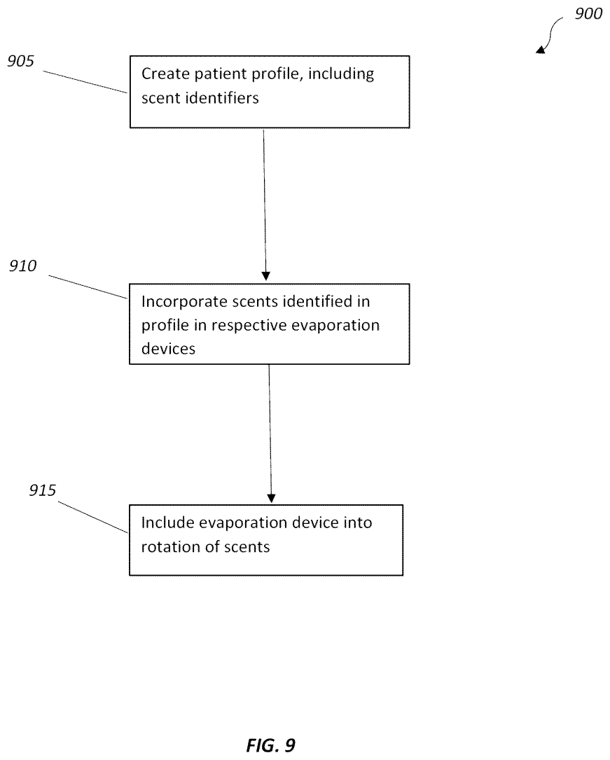

In embodiments, some or all of the scents in a rotation may be created on the basis of personal information provided by the user, where such scents are familiar to the user from his or her past experiences. Such a process is illustrated in FIG. 9. At 905, a scent profile of a user is created, where the scent profile includes identifiers of scents that may be familiar to the user.

Each stage of life, from childhood to senior years, can be accompanied by distinct, memorable moments where scents may evoke lasting emotional connections, making the scent profile deeply personal and reflective of the individual's unique life experiences. A user's scent profile may include identifiers of scents not only familiar to the user but also tied to their personal experiences at various stages of life. For example, as a child in elementary school, it might be the comforting smell of crayons or freshly baked cookies after school. In junior high, it could be the scent of a pine forest from summer camp or the chlorine from swimming lessons. As they move into high school, the smell of fresh-cut grass from sports fields, or the floral scent of a first corsage at a school dance, might evoke strong memories.

In college, perhaps it's the scent of coffee from late-night study sessions, or the aroma of a specific dish that reminds them of home-cooked meals. In their young adult years, moments like a wedding with lavender-scented décor and desserts, or the perfume they wore on that special day, might carry emotional weight. As adults, the scent of baby powder from the early days of parenthood or the familiar aroma of a favorite restaurant may resonate. Finally, in their senior years, scents tied to family traditions, holiday meals, or a favorite flower garden could become meaningful reminders of their journey.

With respect to a user's treatment plan, introducing such familiar, emotionally connected scents, can increase the chances of sparking those memories and feelings. This can be a powerful way to help users recall meaningful moments from their lives, potentially boosting memory and cognitive function. By tapping into these deep personal connections, treatment becomes more engaging and tailored to the individual, which could lead to better therapeutic outcomes. Such scents may be identified through responses to a questionnaire or other mechanisms for the creation of a user profile.

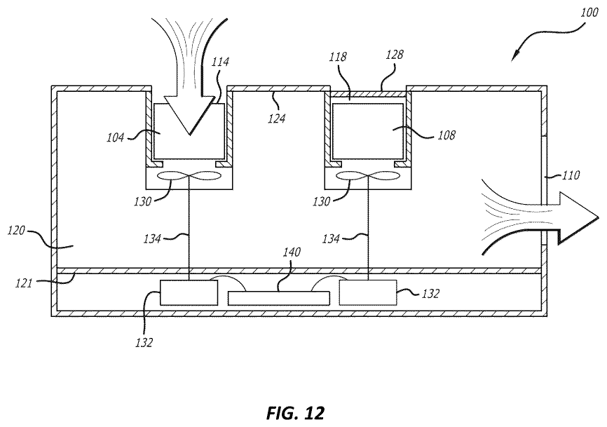

Returning to FIG. 9, at 910, the scents identified in the profile may be incorporated into the device(s) described above. For example, each identified scent may be incorporated into a respective scent presentation device, such as evaporation device 104 (see FIG. 12). At 915, the evaporation devices may be incorporated into the larger device 100 for use in the rotation of scents.

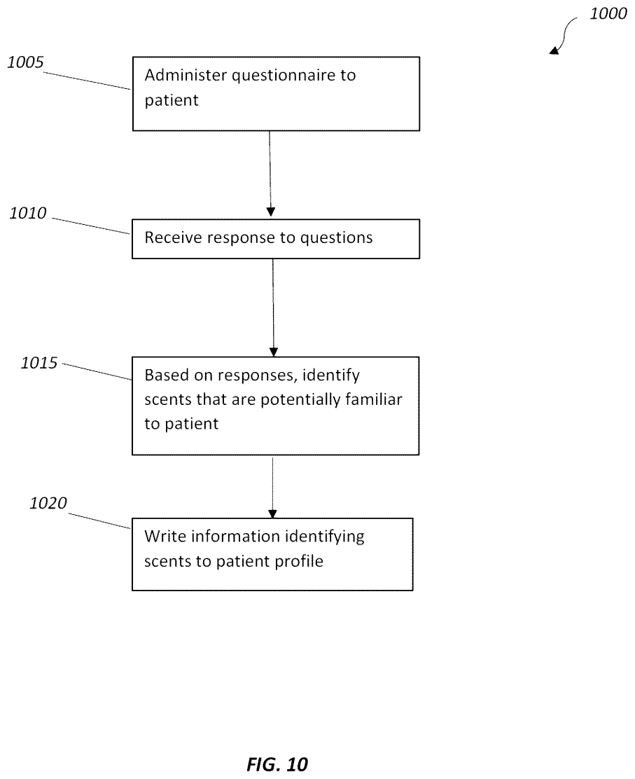

The process of creating a user scent profile is shown in FIG. 10 according to an embodiment. At 1005, a questionnaire may be administered to the user. In various embodiments, such a questionnaire may be administered via a web site user interface, via an application running locally on a user device, or manually via a printed form. An example of such a questionnaire is described in greater detail below. In general, the questionnaire may pose questions related to the user's history, such as his/her ethnic background(s), places where he or she may have lived, and activities that he/she has engaged in previously, as examples without limitation, with a view to ascertaining scents that may be familiar or intimately linked to the user from his/her past.

As previously mentioned, the user's scent profile may include scents that are similar or intimately linked to the user's history. By integrating one or more scents from the user's scent profile into the treatment schedule, strong memories and emotions can be triggered, thereby inducing powerful neural responses. In one embodiment, the order of scents in the treatment schedule may be designed to stimulate memory recall by starting with childhood memories and chronologically moving toward more recent experiences. This gradual timeline allows the brain to strengthen connections across different stages of life, encouraging the retrieval and integration of memories stored over the years. By stimulating and exercising the brain in this sequential way, its ability to bridge long-term memory with more current experiences can be achieved. In this way, reinforcing pathways that are essential for overall memory retention and cognitive function can be created. In another embodiment, the order of scents in the treatment schedule can start with scents associated with more recent memories and progressively moving backward in time to childhood memories.

In another embodiment, the order of scents in the treatment schedule is arranged to deliberately recall memories from the farthest possible point in time and then jumping to recent memories. This back-and-forth stimulation engages opposite areas of the brain—those responsible for long-term memory retrieval and those involved in short-term memory. By alternating between these extremes, the brain is encouraged to work across different neural regions, promoting more balanced neuroplasticity and improving both short-term and long-term memory access. This approach could stimulate cognitive flexibility and enhance the brain's ability to store and retrieve information, ultimately promoting and developing a more resilient brain.
***Uncle Ed: Please draft some claims to put in the claims bank in the spec for the concept above.***

In an embodiment, administration of the questionnaire at 1005 may take the form of interviewing the user or the user's family, where the interview is conducted by another party, such as a family member, a doctor or nurse, or other caregiver. Here, the user may provide responses to questions presented by the caregiver, who then records the user's responses.

At 1010, responses to the questions are received. At 1015, possible prominent scents from the user's past are identified. At 1020, information naming or identifying these scents is written to the user's scent profile.

An example of a questionnaire may include questions such as the following:
User's date of birth?
User's place of birth, including nation and region?
Other places where the user has lived or visited? Time periods when the user was there? For example, favorite vacation place alone, with the family, with friends, etc.
User's ethnic background?
Ethnic background of user's parents or other caregiver(s)?
Favorite foods (present and past) of the user? For example, any type of food associated with a special time in the user's childhood, high school, or when the user met his or her spouse?
Favorite foods (present and past) of the user's parents or other caregiver(s)?
Favorite activities (present and past) of the user?
Favorite activities (present and past) of the user's parents or other caregiver(s)?

In different embodiments, the questionnaire may take different forms and include additional questions, fewer questions, or different questions altogether. The goal of any such questionnaire is to elicit information about scents the user may have been exposed to in the past, especially those associated with key events, favorite memories, life milestones, and places or things with sentimental value that could trigger memory recall and potentially a strong neural response. In an embodiment, the elicited information includes historical experiences and contexts from throughout the user's life, from childhood to present day, or as close to present day as possible in view of any afflictions suffered or pleasant experiences the user has encountered.

For example, the geographic locations where the user was born or where he/she has lived may have corresponding scents with which the user is familiar, e.g., blooming magnolias of the American South, pine forests of the Pacific Northwest, or food smells of New York City's Little Italy. The time at which the user lived in a certain region may also be significant. The food scents of Little Italy may have been more prominent (or otherwise different) in the 1950s than in the 1990s, when that neighborhood had become much smaller.

The user's ethnic background (or that of his/her parents or other caregiver) may also be significant in trying to determine scents familiar to the user. A user who is ethnically Chinese (or had Chinese parents) may recall the smell of incense from holiday celebrations; a user of Indian descent may recall the smell of certain foods or spices. In general, the scent of foods to which a user was exposed, including favorite foods, may be a strong memory, and strongly familiar, for a user.

Likewise the user's current or past hobbies, or those of his parents or caregivers, may have associated scents familiar to the user. If the user is or was interested in certain sports or played such sports, related smells may be familiar to him/her, such as the smell of leather from a football or a baseball glove, or the smell of chalk powder used in gymnastics. Other hobbies have distinct associated scents as well. A user who has enjoyed saltwater fishing may be familiar with scents associated with the ocean, bait, and fish. A user who has enjoyed woodworking may be familiar with the smells of pine or maple, and of wood finishes like varnish, shellac, or paint.

Once responses have been received for questions such as those above, the related scents can be determined. Various mechanisms may be employed to make these determinations. In an embodiment, one or more databases or other data structures may be constructed and used to identify such scents. Such databases may associate possible responses to related scents or information identifying such scents.

For example, a database for regional information may organized as follows:

| REGION | RELATED SCENT(S) |
|---|---|
| Southern US | Magnolia |
| | Wisteria |
| | Gardenia |
| Midwest US | Lily |
| | Lavender |
| | Peony |
| Central Europe | Linden |
| India | Tumeric |
| | Cumin |
| | Cardamon |
| Southeast Asia | Ginger |
| | Star fruit |

The entries above are not meant to be comprehensive and are presented as example data for a database or other data structure pertaining to scents of particular geographic regions.

If a user indicates that he/she has spent time in the southern US, for example, such a database may be accessed and it may then be determined that he/she may have been exposed to scents such as magnolia and wisteria. These scents may then be incorporated into respective evaporation devices for the user's scent rotation.

In another example, a database or other data structure for users' ethnicity information may organized as follows:

| ETHNICITY | RELATED SCENT(S) |
|---|---|
| India | Tumeric |
| | Cardamon |
| | Ginger |
| Southeast Asia | Ginger |
| | Star fruit |
| South America | Pepper |
| | Paprika |
| Mediterranean | Oregano |
| | Rosemary |
| North Africa | Cumin |
| | Corriander |

The entries above are not meant to be comprehensive and are presented as example data for a database or other data structure pertaining to scents of particular ethnicities.

If a indicates that he/she has ethnic roots in the Mediterranean, for example, such a database may be accessed and it may then be determined that he/she may have been exposed to scents such as oregano and rosemary, in light of time spent there and/or the cooking practices of his/her parents. These scents may then be incorporated into respective evaporation devices for the user's scent rotation.

In another example, a database or other data structure for users' hobbies or other activities may organized as follows:

| ACTIVITY | RELATED SCENT(S) |
|---|---|
| Baking | Flour |
| | Chocolate |
| | Cinnamon |
| | Cocoa |
| Baseball | Leather |
| | Sunscreen |
| Gymnastics | Chalk powder |
| Woodworking | Pine |
| | Oak |
| | Varnish |

The entries above are not meant to be comprehensive and are presented as example data for a database or other data structure pertaining to scents of particular activities or hobbies.

If a user indicates that he/she has spent time playing baseball, for example, such a database may be accessed and it may then be determined that he/she may have been exposed to scents such as those of leather and sunscreen. These scents may then be incorporated into respective evaporation devices for the user's scent rotation.

The construction of databases such as those shown above may be done using methods familiar to persons of skill in the art. The data therein may be drawn, for example, from readily available online sources.

### Intensity

The intensity of the scent should be carefully selected based on both its strength and its dissipation rate or longevity. For stronger scents with long-lasting effects, the intensity should be adjusted so that the scent remains present for the duration of the dispersion interval but dissipates in time for the next scent presentation. This ensures that each scent achieves its intended purpose without lingering too long and overlapping with the next scent, maintaining a clear and effective sequence of scent-triggered responses during the treatment. The goal is to strike a balance such that the scent is impactful enough to stimulate a neural response but fades at the right moment for the next scent or break period in the sequence. Optimally, the scent being delivered during each interval 30 will dissipate during the break 40 between the intervals 30 (see FIG. 8). Interestingly, users are not generally affected by scents while they sleep, so the scent being strong enough to interrupt sleep patterns is not a concern. However, as discussed above, olfaction loss occurs with age and should strengthen with treatment. As such, it is an embodiment of the invention to adjust the intensity level prior to the treatment program to a level that is easily detected by the user without being overpowering and interfering with the next scent in the treatment sequence.

### Scent Presentation Devices

A variety of devices are envisioned that could be used to practice the method 10 of the invention. These devices can generally be broken down into two categories-forced air devices and evaporative devices. The forced air devices generally include one or more scent cartridges that are engaged with a machine that pumps air, via one or more fans, bellows, turbine(s), etc., through the cartridge, preferably at a selectable rate such that intensity may be varied. Some embodiments include a plurality of cartridges on a selectable carousel that plays one cartridge at a time. Other devices include a plurality of cartridge engagement assemblies, or “play heads” to allow the cartridges to be played simultaneously.

Another embodiment of the invention includes a device that allows fragrances to be mixed by releasing or “playing” two or more scents simultaneously. The device may also allow the intensity of each scent cartridge to be adjusted by increasing or decreasing the airflow passing through the cartridge. In this way, the “recipes” may be altered by not only adjusting which cartridges are being played, but by adjusting the intensities of each cartridge that is being played. For example, if a “recipe” involves a mixture of cherry and vanilla, the cherry cartridge could be dispersed at a 50% intensity, while the vanilla cartridge could be dispersed at 10% intensity. This would create a different scent that if the vanilla were dispersed at 50% and the cherry were dispersed at 10%, for example.

The evaporative devices may involve scent cartridges, pads or gels that are either contained within a dispensing device, or may be layered such that, once opened, an exposed layer having a first scent begins to evaporate, thereby emitting the first scent. The scent layers may be separated by odorless layers to provide breaks 40 between the scent intervals 30 (see FIG. 8). The dispensing device may have mechanized covers that selectively cover and uncover a scent cartridge or may have heaters associated with each scent cartridge that raises the temperature of the gel to a point that evaporation occurs.

The scent presentation device/system (or dispersion device/system) described herein can, alternatively or in addition, use various types of mechanisms such as a diffusion mechanism, dispersion mechanism, nebulization mechanism, evaporative mechanism, or a combination thereof, for example and without limitation. Hereinafter, the term “dispersion mechanism” can include one or more of the above mechanisms.

The scent presentation device/system can use ultrasonic diffusers, which employ ultrasonic vibrations to create a fine mist from a mixture of water and essential oils. The mist is then released into the air, adding humidity and dispersing the scent.

The scent presentation device/system can use nebulizing diffusers, which work by using pressurized air to atomize pure essential oils, turning them into a fine mist without the use of heat or water.

The scent presentation device/system can use an evaporative diffuser, which uses a fan to evaporate the essential oils from a pad or filter, or simply from oil droplets exposed to the airstream (with no carrier medium). As the oil evaporates, the fan blows the scent into the air. Evaporative diffusion may be suitable for personal use or in volumetrically smaller settings.

The scent presentation device/system can use a heat diffuser to gently heat essential oils, causing them to evaporate and disperse their scent into the air.

The scent presentation device/system can use various types of nebulizers.

The scent presentation device/system can use an ultrasonic nebulizer, which uses high-frequency ultrasonic vibrations to generate a mist from a liquid medication. Such a system may comprise a piezoelectric transducer, which converts electrical energy into mechanical vibrations. These vibrations then propagate through the liquid, creating aerosol droplets. The efficiency of ultrasonic nebulizers in creating a fine mist rapidly is notable, although the thermal effect of the process on medication stability warrants consideration for thermolabile substances.

The scent presentation device/system can use a mesh nebulizer, which uses a mesh or membrane with precisely sized openings, through which the liquid (e.g., oil, water-oil mixture) is forced under pressure, creating a fine mist. This category of systems can be subdivided into active and passive systems. Active mesh nebulizers employ a vibrating element to propel the liquid through the mesh, whereas passive systems rely on the user's inhalation to draw the liquid across. The precision engineering of the mesh ensures consistent droplet size, making these devices highly efficient for drug delivery with minimal residue.

The scent presentation device/system can use a jet nebulizer, which operates on the principle of pressurized air flowing through a narrow orifice, converting a liquid medication into aerosol droplets. This process involves a Venturi system, where the high velocity of air creates a negative pressure that draws the liquid medication from the reservoir, atomizing it into a breathable mist.

The scent presentation device/system can be incorporated into an HVAC system. The dispersion system can be integrated with a building's heating, ventilation, and air conditioning (HVAC) system to disperse scents throughout large areas or entire buildings efficiently. The dispersion system can use cold-air diffusion technology to atomize essential oils into a fine, dry mist that is then distributed evenly through the HVAC ducts. This method is ideal for large spaces such as whole-house, offices, and hotels.

The system can use cold-air diffusers: Similar to HVAC scenting systems but on a smaller scale, cold-air diffusers also use cold-air diffusion technology to atomize essential oils without heat, preserving their therapeutic properties. These standalone units are suitable for medium to large spaces that require consistent scent coverage without connection to the HVAC system.

In an embodiment, a scent presentation system may be integrated into an HVAC system and controlled by smart home technology. This would allow scent presentation to occur according to a schedule that is defined by a user and programmed into the smart home system. Further, a scent dispersion system may be configured to present a scent, pause presentation, then present a different scent (discussed below). In this case, a fan in the HVAC system may be activated during the interval in which presentation is paused, thereby clearing the first scent before the second scent is presented.

It should be noted that the scent presentation system can use any type of aerosolization and evaporation technologies described herein.

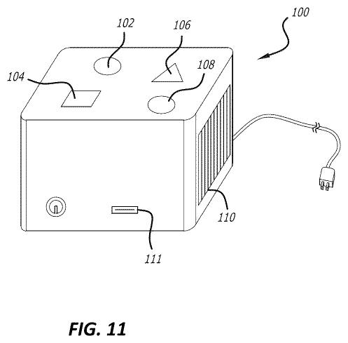

Referring now to FIG. 11, there is shown a first embodiment of a device 100 of the invention. Device 100 is a forced air device that accommodates four cartridges 102, 104, 106 and 108. Each of the cartridges may be a disposable device that allows air to flow through the cartridge to emit a fragrance. Alternatively, in the interest of ecology and cost, the cartridges may be refillable and reusable. More detail about the individual cartridges will be provided below.

Referring to both FIGS. 11 and 12, an embodiment of the device 100 includes a housing 110 with cavities such as cavities 114 and 118 sized and shaped to accommodate cartridges such as 102, 104, 106 and 108. Each cavity 114 and 118 may be equipped with a retractable cover 124 and 128, respectively, such that the cartridges not being used are preserved and aromas therefrom are not deployed.

It is envisioned that each of the cartridges and corresponding cartridges may have a unique shape corresponding to a fragrance family. In this way, a user may place a cartridge belonging to each fragrance family in the device 100. In the example of device 100, the shapes selected are circle, square, triangle and oval for cartridges 102, 104, 106 and 108. Alternatively, each cartridge could be coded, and each cavity could be equipped with a code reader such that the device 100 knows which fragrance is inserted into each cavity.

The housing may have a vent 110 formed in one wall through which the fragrance from the cartridges is emitted. The sidewall was selected as most users will place the device 100 on a bedside table. The vent 110 may then be situated such that it faces the user.

Any such cartridge, whether it contains the scent in a liquid form or incorporates the scent in a semi-solid or solid medium such as a wax, may be viewed as a container for the scent.

Internally, the device as shown in FIG. 12 may include a fan or fans 130 situated beneath the cavities. The fan 130 may be powered by a motor 132 and connected to the motor with a shaft 134. A control board 140 may include logic that activates/deactivates each motor along with logic functions discussed in detail below. In the embodiment having one fan, flow through the individual cartridges may be controlled by the covers 124 and 128.

The device 100 is shown as drawing air downward through the cartridges and into a central housing cavity 120. The air is forced to exit through the vent 110 because the vent 110 is the only exit for the cavity, considering that the covers are closed on cavities not being used and that fans are running on each cavity with an open cover. This design allows the central housing cavity 120 to be used as a mixing chamber in the event that more than one fragrance cartridge is being used simultaneously. In order to protect the electronic componentry, such as the motors 132 and the control board 140, a barrier 121 is provided, insulating these components from the central housing cavity 120.

One skilled in the art will realize that the device 100 may be designed such that air flow is reversed, without departing from the spirit of the invention. A reversed air flow could be accomplished by merely reversing the direction of the fans 130. This design may be advantageous in that there would be less risk of residue building up in the cavity 120.

A control device or module, such as control board 140, may be electrically connected to the fan motors 132 and may include a wireless connection technology, such as Bluetooth® that can be connected to an electronic device such as a smartphone or reader. An application, downloadable onto the device, may include control logic that allows a user to customize a treatment schedule. Alternatively, a physician may create a schedule 20 that is downloadable by the user.

The control module may control dispersion of a first scent for a predetermined first duration, employ a break period after the predetermined first duration, and disperse a second scent for a predetermined second duration after the break period.

The break period can be based on the type of scent just dispersed, how strong the scent is, and/or where it is located on a scent pyramid model.

The pyramid may be divided into three main layers or notes, each representing a specific phase of the fragrance's life. These layers are:

Top Notes (Head Notes): These are the initial scents that are perceived immediately upon application of a perfume, for example. Top notes are usually light, fresh, and volatile, evaporating quickly. They give the first impression of the fragrance but last for a short duration, typically ranging from a few minutes to an hour. Common top notes include citrus (like lemon and bergamot), light fruits (such as berries and grapes), and herbs (like lavender and rosemary).

Middle Notes (Heart Notes): After the top notes dissipate, the middle notes become noticeable. They are considered the heart of the fragrance, representing the main elements of the scent composition. Middle notes are more mellow and rounded than top notes and are perceived during the majority of the fragrance's wear on the skin. They typically last longer than top notes but are less volatile, usually lasting several hours. Common middle notes include floral (like rose and jasmine), spice (such as cinnamon and cardamom), and fruit scents (such as peach and plum).

Base Notes: These are the last to develop and the longest-lasting notes. Base notes give depth and solidity to the fragrance, lingering for an extended time after the top and middle notes have faded. They are usually rich, heavy, and deep scents that anchor the fragrance, preventing the lighter notes from evaporating too quickly. Common base notes include woodsy scents (like sandalwood and cedarwood), resins (such as amber and myrrh), musk, and vanilla.

In some embodiments, the break period can be longer for base notes than top notes. The break period can also be based on the room size, number of windows, available air ducts, type of scents, or a combination thereof.

The control module may comprise programmable logic circuit (PLC) that allows for the customization of operational sequences. The PLC may be programmed to initiate a control signal to activate a dispersion mechanism for a specified first duration.

Upon receiving a control signal, the dispersion mechanism may operate for a predetermined first duration, performing its designated function. The duration is programmable and can be set according to the requirements of a specific application.

Predefined Break Period: After operating for a first duration, the PLC may initiate a break period. This period is of a predefined or programmable length during which dispersion mechanisms are inactive. This break period facilitates the dissipation of the first scent, ensuring it can no longer be detected (via smell).

After the break period, the PLC may generate a second control signal to activate the same or different dispersion mechanism to disperse a second scent. The same or different dispersion mechanism then operates for a predetermined second duration. In the embodiment where the same dispersion mechanism is employed, liquid (e.g., oil, water-oil mixture) having a first scent is transferred into a dispersion chamber where the dispersion mechanism is configured to disperse any liquid transferred into the dispersion chamber. In this embodiment, the dispersion device/system can have multiple scent reservoirs and a single dispersion chamber. In another embodiment, the scent presentation device/system can have multiple dispersion mechanisms. In this embodiment, each scent reservoir can have its own dispersion mechanism and as such a centralized dispersion chamber is not needed. However, the dispersion system can include a combination of scent reservoirs having their own dispersion mechanisms and scent reservoirs with a shared dispersion chamber.

Incorporated within the PLC may be timing and sequencing logic algorithms that ensure the precise control of the activation periods and the break period. This logic is programmable to accommodate various sequences, durations, and operational requirements.

The control module may include an interface for programming and adjusting the sequence parameters, durations, and break periods. This interface may be a physical set of controls on the module or a software application communicating with the module via wired or wireless connections (e.g., over-the-air updates).

The device 100 may also interact with external appliances such as fans and air filters. In an embodiment, the device 100 may be in wireless communication, through control board 140 and components thereof, with a high efficiency particulate air (HEPA) filter located in the vicinity of the device 100 and the user. The HEPA filter may be activated, under the control of device 100, during break periods. With this arrangement, the air may be cleared of a previous scent before the next scent is presented, at which time the filter may be turned off as directed by device 100.

The application provides various controls and options that may be incorporated into the treatment schedule, including scent rotation, intensity, timing, reminders, scent shuffle, and the like. The application preferably tracks the scent rotation to assess which scents may have been played repeatedly over the course of the treatment program. These scents can be marked by the application as losing novelty and can thus be taken out of the rotation, or mixed with another scent the next time they are used.

The application can be wirelessly connected to a variety of biometric sensors listed above, such that the schedule becomes dynamically-timed. For example, the user may don a heart rate monitor when going to bed. The activation of the heart rate monitor is sensed by the application and the application begins to monitor the sleep stage of the user to determine the timing of the treatment schedule 20, as discussed above.

Alternatively, if a biometric sensor is not used, the handheld electronic device itself could be used by the control board to indicate the beginning of a treatment schedule 20. For example, the device 100 may include a USB port 111 that is connected to the control board. The user could use this USB port to charge their phone during the evening. Plugging the phone into the device 100 could be used as an indication that the treatment schedule 20 should commence. Ease of use is thus maximized as the disruption in routine is minimized, as most people are accustomed to charging their smart phones in the evening.

The control board 140 is also used to track the usage of the cartridges. Depending on the construction of the cartridge, the application can be used to provide indications to the user that a particular cartridge needs to be replaced.

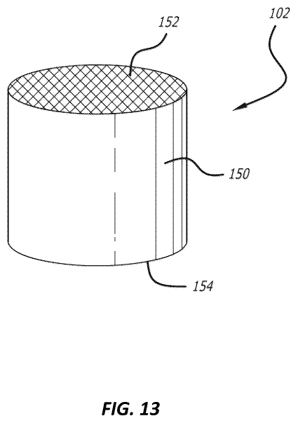

FIG. 13 shows an embodiment of a cartridge 102 of the invention. This embodiment is a disposable embodiment and includes a housing 150 with a top 152 and a bottom 154. The top 152 and bottom 154 are vented such that air may pass through. The cartridge 102 may be shipped with a removable film (not shown) covering the top 152 and bottom 154 until the cartridge is ready to be used, thus preserving the odorant contained therein.

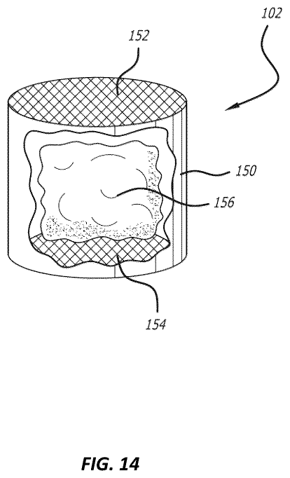

FIG. 14 shows a cutaway view of the cartridge 102, allowing an absorbent odorant pad 156 to be seen. The pad 156 may be formed as a wad, such as a cotton ball or polyester fiberfill wad, and should be loosely packed such that air may flow through it. Alternatively, the pad 156 may be slightly smaller than an interior of the cartridge such that air may flow around the pad 156. In this embodiment, a denser wadding material may be used that might hold more odorant than an embodiment that requires the air to flow through the pad 156.

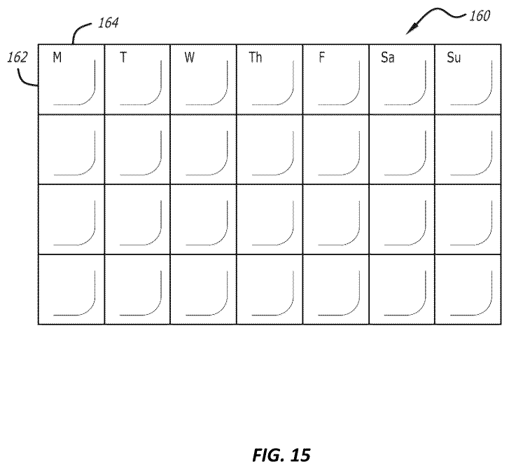

In one embodiment of the cartridge 150 of FIG. 14, the top 152 is removable and replaceable. This embodiment may reduce plastic waste and costs. Rather than replacing the entire cartridge, odorant pads 156 could be provided or sold individually or in packages containing multiple packages. FIG. 15 shows a package 160 of pads 156 arranged such that each row 162 represents a different scent family and each column 164 thus represents a different day of the week.

As introduced above, one aspect of the invention provides evaporative devices that involve scent cartridges, pads or gels that are either contained within a dispensing device, or may be layered such that, once opened, an exposed layer having a first scent begins to evaporate, thereby emitting the first scent. The scent layers may be separated by odorless layers to provide breaks 40 between the scent intervals 30 (see FIG. 8).

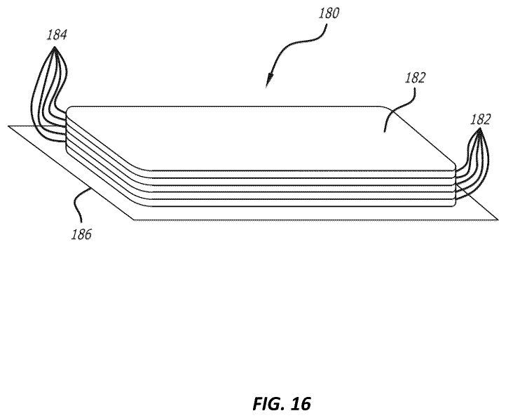

One embodiment 180 of an evaporative device is shown in FIG. 16. This device is a gel tab consisting of a plurality of odorant layers 182 separated by odorless layers 184. In this example there are six odorant layers 182 separated by five odorless layers. The odorant layers are designed to evaporate at a desired rate such that each odorant layer represents an interval 30. The odorless layers are designed to evaporate at a desired rate such that each odorless layer represents a break 40. The desired timing of each layer may be controlled by the thickness of the layer during the manufacturing process. The odorless layers 184 would then be thinner than the odorant layers 182. The gel tabs 180 are provided on a substrate 186 such that only the top layer evaporates.

The gel tab 180 provides a base for a variety of delivery mechanisms encompassed in the invention. Additionally, it is envisioned that the gel tab 180 could shaped such that it may be removed from the substrate 186 and applied like tap directly to the upper lip or chin of the user. In this embodiment, it may be desired to include a odorless layer 184 as the top layer such that the user has a few minutes to fall asleep prior to experiencing the odorants.

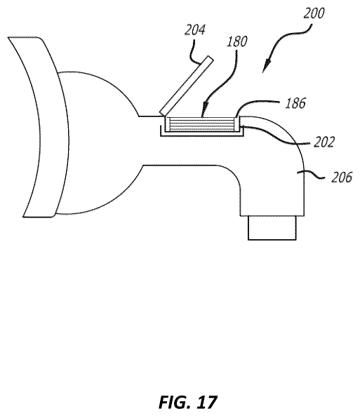

FIG. 17 shows a CPAP mask 200 that has been modified slightly to accommodate a gel tab 180. The mask 200 has a nozzle 206 that connects to the gas delivery tube of a typical CPAP machine (not shown). The nozzle 206 includes a compartment 202 with a lid 204 that can be opened so that a gel tab 180 may be placed therein. The tab 180 is placed such that the substrate 186 is on the top. During use, the gas flowing through the nozzle 206 from the CPAP machine passes under the bottom, exposed layer of the tab 180 and is inhaled by the user. In the morning, the empty substrate 186 is discarded.

Other devices are envisioned that utilize the gel tab 180 including, but not limited to, plug-in style air fresheners, non-CPAP masks, pendants and other wearable devices, nose plugs, heat-activated devices, diffusers, etc.

Alternatively, a device such as that described in U.S. patent application Ser. No. 18/423,755, filed Jan. 26, 2024 and incorporated herein by reference in its entirety, may also be used for the dispersion of scents in the manner described here.

The scent presentation device/system may have a plurality of reservoirs, each containing a different scent. The plurality of reservoirs can also include one or more scent neutralizers.

In some embodiments, the device/system can have multiple reservoirs and a dispersion reservoir having a dispersion mechanism configured to disperse any liquid in the dispersion reservoir. The dispersion reservoir can be centrally located to the multiple scent reservoirs. In operation, liquid from one of the scent reservoirs can be transferred (e.g., pump, gravity) into the dispersion reservoir. Once the transfer process is completed, the dispersion mechanism can be activated to disperse the liquid therein.

In some embodiments, liquid from two or more reservoirs can be transferred to the dispersion reservoir for dispersion. In this way, a unique blend of aromas can be created using two or more scent liquids.

The dispersion reservoir can be fluidically coupled to an air dispersion chamber where an airstream is provided by a fan or other means.

In some embodiments, each scent reservoir incorporates its own dispersion mechanism and operates as its own dispersion reservoir. Each scent reservoir can be fluidically coupled to an air chamber where an airstream is provided. The air chamber can be a common air chamber or separate, with each dispersion reservoir having its own air chamber.

Liquid from each reservoir can be transferred to the dispersion reservoir using a pump or by gravity. In the gravity embodiment, the scent reservoir can be located at a higher position than the dispersion reservoir.

In some embodiments, the dispersion device/system can dispense droplets of the liquid (e.g., essential oil, water-oil mixture) into a dispersion chamber where the droplets are exposed to an airstream using various means such as a fan, convection, pressure differential system, etc. The dispersion chamber may also include a dispersion plate configured to disperse or evaporate the droplets. The dispersion plate can employ aerosolization or evaporation mechanism as described herein. For example, the dispersion plate can use an ultrasonic transducer to disperse the droplets. Alternatively, the dispersion plate can use a heat source to speed up the evaporation process.

Various mechanisms can be employed to accurately disperse liquid droplets. The dispersion device/system can employ any of the droplets dispensing mechanisms below.

Piezoelectric droplet ejection leverages the properties of piezoelectric materials to precisely eject liquid droplets, a technique widely adopted in inkjet printing. Similarly, microfluidic devices manipulate fluids within micro-scale channels to produce uniform droplets.

Syringe pumps and micro-pumps offer controlled liquid displacement, enabling precise dosing in medical and laboratory contexts. Electrospray and electrospinning techniques apply high voltage to liquids, creating charged droplets or fine fibers, utilized in coating and material science applications.

### EXAMPLES

The following examples are presented to illustrate the present invention and to assist one of ordinary skill in making and using the same. The examples are not intended in any way to otherwise limit the scope of the invention.

### Example 1: Environmental Enrichment in Inner-City Classrooms

This Example demonstrates enhancing brain responses with multiple olfactory stimulation in school children. Children in an inner-city school were exposed to 30 minutes of olfactory stimulation daily in the classroom using essential oil fragrances, one per day, rotating through 5 different scents (n=25 children), for 3 months. Another group of 23 children was left without such stimulation, serving as controls. Stroop test scores were monitored over that period. This test assesses cognitive processing speed, attention capacity, and executive function. In addition, the Stroop test has a strong correlation with IQ, general behavior, and school performance (Imbrosciano and Berlach, 2005).

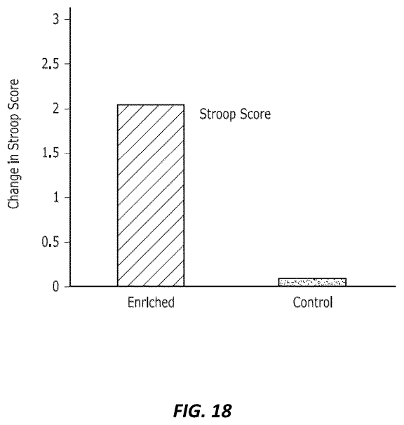

As shown in FIG. 18, olfactory-enriched children had more than 10 times the improvement observed in controls on that test. The classes were team taught by the same teachers, making it unlikely that this phenomenon was due to differences in the teaching ability of their teachers. While there have been many attempts to improve cognitive outcomes for children in elementary school, such attempts have generally been unsuccessful in increasing their cognitive capacity.

### Example 2: Environmental Enrichment for Older Adults

This example demonstrates the positive impact of olfactory stimulation on the cognitive ability of older adults. The older adults were tested using standard, validated cognitive assessments, and then retested after they had received daily olfactory stimulation. The stimulated older adults had improvements that were 181%, 268% and 1,258% better than controls.

Older adults were given a set of pleasant scents and asked to expose themselves to the scents using a provided nebulizer for 30 min in the morning and 30 min in the evening every day for 6 months. Forty older adults (60-75 years old) gave themselves patterned olfactory stimulation at home, or were assigned to a group that did not experience increased olfactory stimulation. Three tests of cognition were administered at the start of the trial, and then again after 6 months.

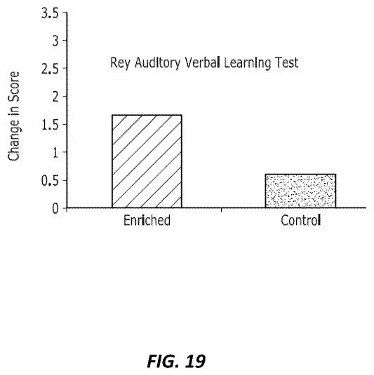

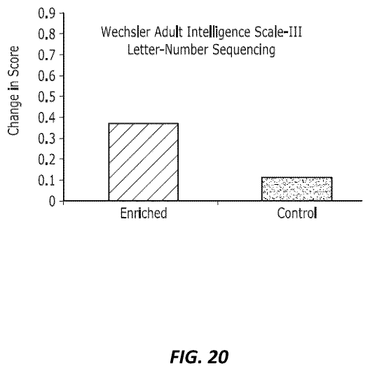

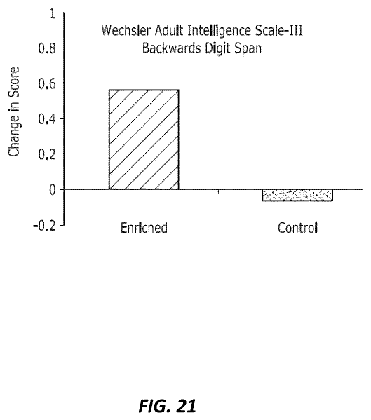

In a test that reveals verbal learning and memory (Rey Auditory Verbal Learning), olfactory-enriched older adults performed 181% better than controls (FIG. 19). In a test of sequential processing, mental manipulation, attention, concentration, memory span, and short-term auditory memory (WAIS III Letter-Number Sequencing), the enriched group had a 268% advantage over controls (FIG. 20). Finally, in a test of rote learning and memory, attention, encoding, and auditory processing, working memory, transformation of information, mental manipulation, and visual-spatial imaging (WAIS III Backwards Digit Span), the enriched group performed 1,258% better than controls (FIG. 21).

These cognitive benefits are about 1-2 orders of magnitude better than computer games, direct brain stimulation, exercise or social interactions (Clememson and Stark, 2015; Ezzyat, et al., 2018; Hertzog, et al. 2009; Kirk-Sanchez and McGough, 2014; Kucewisc, et al., 2018).

The invention described herein may include a method of improving cognitive function of a user comprising: establishing a treatment schedule including a plurality of intervals separated by breaks; and inputting the treatment schedule into a scent-delivery device such that the device delivers one or more of a plurality of scents during each interval, and stops the delivery of the scent at the end of the interval, the plurality of scents comprising at least one scent identified in a profile of said user where the at least one identified scent is familiar to the user from a past experience. In an embodiment, scents delivered during consecutive intervals are sufficiently distinct from each other so as to be distinguishable by said user. In an embodiment, no scent is repeated more than once during the treatment schedule. In an embodiment, this method further comprises repeating the treatment schedule for a predetermined treatment length to improve far transfer effects for the user.

In an embodiment, this method further comprises creation of the profile, wherein creation of the profile comprises: posing questions to the user, the questions relating to one or more of the ethnicity of the user, locations where the user has lived previously, and activities in which the user has been previously engaged; receiving responses from the user; based on the received responses, identifying scents corresponding to the responses; and including, in the profile, information specifying the identified scents. The identifying of the scents corresponding to the responses may comprise accessing one or more databases that relate identifiers of scents to potential responses to the posed questions; and reading any identifiers of scents that correspond to the received responses. In an embodiment of this method, establishing the treatment schedule including the plurality of intervals separated by breaks comprises scheduling at least three intervals separated by breaks.

In an embodiment of this method, inputting the treatment schedule into a scent-delivery device comprises wirelessly connecting the device to a handheld electronic device having an application that allows the treatment schedule to be uploaded from the handheld electronic device to the scent-delivery device.

In an embodiment of this method, establishing a treatment schedule comprises basing an initiation of the treatment schedule on an activity signifying that a user is going to bed. The activity may comprise communicatively coupling a handheld device to the scent-delivery device. The activity may comprise wirelessly coupling a biometric sensor to the hand-held device, providing data that indicates the user has begun a sleep cycle.

In an embodiment of this method, the scents identified in the profile comprise scents experienced by the user in a time interval between childhood and the initiation of treatment. In an embodiment, the scents may be delivered in a sequence corresponding to a chronological order in which the user experienced the plurality of scents during his/her lifetime. The scents may alternatively be delivered in a sequence that is non-chronological with respect to an order in which the user experienced the plurality of scents during his/her lifetime.

The invention described herein may include a device for providing cognitive-improvement olfactory therapy, the device comprising: a housing defining at least one cavity, each of said at least one cavity sized to receive a removable scent cartridge, and a vent; at least one fan adjacent the at least one cavity and capable of moving air through said at least one cavity; at least one motor configured to activate the at least one fan; a control board that controls the at least one motor according to a treatment schedule that includes a plurality of intervals separated by breaks, and initiates the treatment schedule at a start event, wherein during an interval, the at least one fan is activated such that air is moved through the vent and an associated at least one cavity, thereby emitting a scent when a scent cartridge is present in the at least one cavity, wherein the scent is one of a plurality of scents comprising at least one scent identified in a profile of a user where the at least one identified scent is familiar to the user from a past experience; and wherein during a break, the at least one fan is inactive. In an embodiment, this device further comprises moveable covers associated with each of said cavities and mechanized to move between an open position to a closed position, said movement of said covers controlled by said control board.

In an embodiment of this device, the start event comprises a time of day. In an embodiment of this device, the start event comprises data received from a biometric sensor indicating that a user has begun a sleep cycle. An embodiment of this device further comprises a personal electronic device wirelessly connected to said control board, wherein said personal electronic device provides said treatment schedule to said control board.

In an embodiment of this device, the start event is determined by the personal electronic device.

In an embodiment of this device, the scents identified in the profile comprise scents experienced by the user in a time interval between childhood and the initiation of treatment.

The invention described herein may include a method of improving cognitive function of a user comprising: providing a plurality of scents categorized into scent families, each family including scent sub-groups, according to a fragrance wheel arranged such that adjacent sub-groups have similarities, the plurality of scents comprising at least one scent identified in a profile of said user where the at least one identified scent is familiar to the user from a past experience; and establishing a treatment schedule including exposure of the user to a plurality of intervals separated by breaks, wherein a scent is administered to the user during an interval and no scent is administered during a break, wherein scents delivered during consecutive intervals are sufficiently distinct from each other so as to be distinguishable by said user. In an embodiment of this method, no scent is repeated more than once during the treatment schedule. An embodiment of this method further comprises repeating the treatment schedule each day for a predetermined treatment length to improve far transfer effects for the user.

An embodiment of this method further comprises creation of the profile, wherein the creation of the profile comprises: posing questions to the user, the questions relating to one or more of the ethnicity of the user, locations where the user has lived previously, and activities in which the user has been previously engaged; receiving responses from the user; based on the received responses, identifying scents corresponding to the responses; and including, in the profile, information specifying the identified scents. The identifying of the scents corresponding to the responses may comprises: accessing one or more databases that map identifiers of scents to potential responses to the posed questions; and reading any identifiers of scents that correspond to the received responses.

In an embodiment of this method, administering the schedule to the user comprises using a device that automatically emits said scents according to said schedule. The using of the device may comprise programming the device to begin the treatment schedule a predetermined amount of time after the user has gone to bed.

In an embodiment of this method, the scents are presented to the user according to the treatment schedule while the user is sleeping.

In an embodiment of this method, the scents identified in the profile comprise scents experienced by the user in a time interval between childhood and the initiation of treatment.

The invention described herein may include a method for scent dispersion, the method comprising: dispersing a first scent from a first scent container for a first duration, the first scent having a first characteristic; providing a first dispersion break period after the first duration, wherein the first duration and the first dispersion break are based on the first characteristic; and dispersing a second scent from a second scent container for a second duration, the second scent having a second characteristic, wherein the second duration is based on the second characteristic. This method may further comprise providing a second dispersion break period after the second duration, wherein the second dispersion break period is based on the second characteristic.

In this method, the first or second characteristics may comprise notes of the respective first or second scents. The first duration of a top note scent may be longer than a duration of a scent with a middle or base note, and the first dispersion break period after the first duration of the top note scent may be shorter than the second dispersion break period for the second duration of a scent with middle or base note.

In an embodiment of this method, the first duration of a middle note scent may be longer than a duration of a scent with a base note. In an embodiment, the first dispersion break period after the first duration of the middle note scent may be shorter than the second break period for the second duration of the scent with the base note.

The invention described herein may include a scent dispersion system, comprising: means for dispersing each of a plurality of scents for a respective duration, where the dispersions are each followed by a dispersion break period in which no scent is dispersed; a control module; and connectivity to an air filter, wherein the control module is configured to control activation of the air filter, to activate the air filter during the dispersion break periods.

The invention described herein may include a scent presentation device, the device comprising: a plurality of scent containers each configured to contain a liquid; at least one dispersion mechanism configured to disperse the liquids into an environment; and a control module configured to cause the at least one dispersion mechanism to disperse a first liquid from a first scent container of the plurality of scent containers for a first duration, and to disperse a second liquid from a second scent container of the plurality of scent reservoirs for a second duration, wherein at least one of the scents produced from the liquids are chosen for dispersion after being identified in a profile of a user where the scent is familiar to the user from a past experience. In an embodiment, the scents identified in the profile comprise scents experienced by the user in a time interval between childhood and the initiation of treatment.

### REFERENCES

Adams D R, Kern D W, Wroblewski K E, Mcclintock M K, Dale W, Pinto J M. (2018). Olfactory dysfunction predicts subsequent dementia in older U.S. adults. J Am Geriatr Soc. 66:140-144.
Altundag A, Cayonu M, Kayabasoglu G, Salihoglu M, Tekeli H, Saglam O, Hummel T. (2015), Modified olfactory training in users with postinfectious olfactory loss. Laryngoscope. 125:1763-1766.
Arendash G W, Garcia M F, Costa D A, Cracchiolo J R, Wefes I M, Potter H. (2004). Environmental enrichment improves cognition in aged Alzheimer's transgenic mice despite stable beta-amyloid deposition. Neuroreport. 15:1751-1754.
Aronoff E, Hillyer R, Leon M. (2016). Environmental enrichment therapy for autism: outcomes with increased access. Neural Plasticity, vol. 2016, Article I D 2734915, 23 pages.
Arranz L, De Castro N M, Baeza I, Giménez-Llort L, De la Fuente M. (2011). Effect of environmental enrichment on the immunoendocrine aging of male and female triple-transgenic 3×Tg-AD mice for Alzheimer's disease. J Alzheimer's Dis. 25:727-737.
Basak C, Boot W R, Voss M W, Kramer A F. (2008). Can training in a real-time strategy videogame attenuate cognitive decline in older adults? Psychol Aging. 23:765-777.
Berardi N, Braschi C, Capsoni S, Cattaneo A, Maffei L. (2007). Environmental enrichment delays the onset of memory deficits and reduces neuropathological hallmarks in a mouse model of Alzheimer-like neurodegeneration. J Alzheimers Dis. 11:359-370.
Bitter T, Brüderlea J, Gudziol H, Hartmut Peter Burmeister H P, Gaser C, Guntinas-Lichius O. (2010b). Gray and white matter reduction in hyposmic subjects-A voxel-based morphometry study. Brain Res. 1347:42-47.
Bitter T, Gudziol H, Burmeister H P, Mentzel H-J, Guntinas-Lichius O, Gaser C. (2010a). Anosmia leads to a loss of gray matter in cortical brain areas. Chem Senses 35:407-415.
Bitter T, Siegert F, Gudziol H, Burmeister H P, Mentzel H J, Hummel T, Gaser C, Guntinas-Lichius O. (2011). Gray matter alterations in parosmia. Neuroscience. 177:177-182.
Boudjarane M A, Grandgeorge M, Marianowski R, Misery L, Lemonnier É. (2017). Perception of odors and tastes in autism spectrum disorders: A systematic review of assessments. Autism Res. 10:1045-1057.
Choi J S, Hur K, Chow M, Shen J, Wrobel B. (2018). Olfactory dysfunction and cognition among older adults in the United States. Int Forum Allergy Rhinol. [Epub ahead of print]
Conti M Z, Vicini-Chilovi B, Riva M, Zanetti M, Liberini P, Padovani A, Rozzini L. Odor identification deficit predicts clinical conversion from mild cognitive impairment to dementia due to Alzheimer's disease. Arch Clin Neuropsychol. 28:391-399.
Clemenson G D, Stark CEL. (2015). Virtual environmental enrichment through video games improves hippocampal-associated memory. J. Neurosci. 35:16116-16125.
Costa D A, Cracchiolo J R, Bachstetter A D, Hughes T F, Bales K R, Paul S M, Mervis R F, Arendash G W, Potter H. (2007). Enrichment improves cognition in A D mice by amyloid-related and unrelated mechanisms. Neurobiol Aging. 28:831-844.
Damm M, Pikart L K, Reimann H, Burkert S, Göktas Ö, Haxel B, Frey S, Charalampakis I, Beule A, Renner B, Hummel T, Hüttenbrink K B. (2014). Olfactory training is helpful in postinfectious olfactory loss: a randomized, controlled, multicenter study. Laryngoscope. 124:826-831.
Devanand D P, Michaels-Marston K S, Liu X, Pelton G H, Padilla M, Marder K, Bell K, Stern Y, Mayeux R. (2000). Olfactory deficits in users with mild cognitive impairment predict Alzheimer's disease at follow-up. Am J Psychiatry. 157:1399-1405.
Devanand D P, Tabert M H, Cuasay K, Manly J J, Schupf N, Brickman A M, Andrews H, Brown T R, DeCarli C, Mayeux R. (2010). Olfactory identification deficits and MCI in a multi-ethnic elderly community sample. Neurobiol Aging. 31:1593-600.
Dong J, Pinto J M, Guo X, Alonso A, Tranah G, Cauley J A, Melissa Garcia, Satterfield S, Huang X, Harris T, Mosley T H, Chen H. (2017). The prevalence of anosmia and associated factors among U.S. black and white older adults. J Gerontol A Biol Sci Med Sci. 72:1080-1086.
Doty R L, Deems D A, Stellar S. (1988). Olfactory dysfunction in Parkinsonism: a general deficit unrelated to neurologic signs, disease stage, or disease duration. Neurology. 38:1237-1244.
Doty R L, Shaman P, Applebaum S L, Giberson R, Siksorski L, Rosenberg L. (1984). Smell identification ability: changes with age. Science. 226:1441-1443.
Endevelt-Shapira Y, Perl O, Ravia A, Amir D, Eisen A, Bezalel V, Rozenkrantz L, Mishor E, Pinchover L, Soroka T, Honigstein D, Sobel N. (2018). Altered responses to social chemosignals in autism spectrum disorder. Nat Neurosci. 21:111-119.
Ezzyat Y, Wanda P A, Levy D F, Kadel A, Aka A, Pedisich I, Sperling M R, Sharan A D, Lega B C, Burks A, Gross R E, Inman C S, Jobst B C, Gorenstein M A, Davis K A, Worrell G A, Kucewicz M T, Stein J M, Gorniak R, Das S R, Rizzuto D S, Kahana M J. (2018). Closed-loop stimulation of temporal cortex rescues functional networks and improves memory. Nat Commun. 9:365.
Geißler K, Reimann H, Gudziol H, Bitter T, Guntinas-Lichius O. (2014). Olfactory training for users with olfactory loss after upper respiratory tract infections. Eur Arch Otorhinolaryngol. 271:1557-1562.
Gopinath B, Sue C M, Kifley A, Mitchell P. (2012). The association between olfactory impairment and total mortality in older adults. J Gerontol. Series A, 67A: 204-209.
Haehner A, Tosch C, Wolz M, Klingelhoefer L, Fauser M, Storch, A, Reichmann H, Hummel T. (2013) Olfactory training in users with Parkinson's disease. PLOS ONE. 8: e61680.
Hertzog C, Kramer A F, Wilson R S, Lindenberger U. (2008) Enrichment effects on adult cognitive development: can the functional capacity of older adults be preserved and enhanced? Psychol Sci Public Interest. 9:1-65.
Hoffman H J, Rawal S, Li C-M, Duffy V B. (2016). New chemosensory component in the U.S. National Health and Nutrition Examination Survey (NHANES): first-year results for measured olfactory dysfunction. Rev Endocr Metab Disord. 17:221-240
Hoffmann K, Sobol N A, Frederiksena K S, Beyerb N, Vogela A, Vestergaardd K, Brændgaarde H, Gottrupe H, Lolkf A, Wermuthf L, Jacobseng S, Laugesenh L P, Gergelyffyh R G, Høghi P, Bjerregaardj E, Andersena B B, Siersmak V, Johannsena P, Cotman C W, Waldemara G, Hasselbalcha S G. (2016). Moderate-to-high intensity physical exercise in users with Alzheimer's disease: a randomized controlled trial. J Alzheimers Dis. 50:443-453.
Hummel T, Rissom K, Reden J, Hähner A, Weidenbecher M, Hüttenbrink K-B. (2009), Effects of olfactory training in users with olfactory loss. Laryngoscope. 119:496-499.
Jankowsky J L, Melnikova T, Fadale D J, Xu G M, Slunt H H, Gonzales V, Younkin L H, Younkin S G, Borchelt D R, Savonenko A V. (2005). Environmental enrichment mitigates cognitive deficits in a mouse model of Alzheimer's disease. J Neurosci. 25:5217-5224.
Kerr B, Silva P A, Walz K, Young J I. (2010). Unconventional transcriptional response to environmental enrichment in a mouse model of Rett syndrome. PLOS ONE. 5, e11534.
Kirk-Sanchez N J, McGough E L. (2014). Physical exercise and cognitive performance in the elderly: current perspectives. Clin Interven Aging. 9:51-62.
Klimova B. (2016). Computer-based cognitive training in aging. Front Aging Neurosci. 8:313.
Kollndorfer K, Jakab A, Mueller C, Trattnig S, Schopf V. (2015). Effects of chronic peripheral olfactory loss on functional brain networks. Neuroscience. 310:589-599.
Kondo M, Gray L J, Pelka G J, Christodoulou J, Tam P P, Hannan A J. (2008). Environmental enrichment ameliorates a motor coordination deficit in a mouse model of Rett syndrome—Mecp2 gene dosage effects and BDNF expression. Eur J Neurosci. 27, 3342-3350.
Konstantinidis I, Tsakiropoulou E, Bekiaridou P, Kazantzidou C, Constantinidis J. (2013). Use of olfactory training in post-traumatic and postinfectious olfactory dysfunction. Laryngoscope. E85-E90.
Konstantinidis I, Tsakiropoulou E, Constantinidis J. (2016). Long term effects of olfactory training in users with postinfectious olfactory loss. Laryngoscope. 54:170-175.
Kucewicz M T, Berry B M, Miller L R, Khadjevand F, Ezzyat Y, Stein J M, Kremen V, Brinkmann B H, Wanda P, Sperling M R, Gorniak R, Davis K A, Jobst B C, Gross R E, Lega B, Van Gompel J, Stead S M, Rizzuto D S, Kahana M J, Worrell G A. (2018). Evidence for verbal memory enhancement with electrical brain stimulation in the lateral temporal cortex. Brain. [Epub ahead of print]
Lacaria M, Spencer C, Gu W, Paylor R, Lupski J R. (2012). Enriched rearing improves behavioral responses of an animal model for CNV-based autistic-like traits. Human Mol Genet. 21, 3083-3096.
Lafaille-Magnan M-E, Poirier J, Etienne P, Tremblay-Mercier J, Frenette J, Rosa-Neto P, Breitner J C S, for the PREVENT-A D Research Group. (2017). Odor identification as a biomarker of preclinical A D in older adults at risk. Neurology. 89:327-335.
Lazarov O, Robinson J, Tang Y P, Hairston I S, Korade-Mirnics Z, Lee V M, Hersh L B, Sapolsky R M, Mirnics K, Sisodia S S. (2005). Environmental enrichment reduces Abeta levels and amyloid deposition in transgenic mice. Cell. 120:701-713.
Li W, Howard J D, Gottfried J A. (2010). Disruption of odour quality coding in piriform cortex mediates olfactory deficits in Alzheimer's disease. Brain. 133:2714-2726.
Liu G, Zong G, Doty R L, Sun Q. (2016). Prevalence and risk factors of taste and smell impairment in a nationwide representative sample of the U S population: a cross-sectional study. BMJ Open. 6: e013246.
Lonetti G, Angelucci A, Morando L, Boggio E M, Giustetto M, Pizzorusso T. (2010). Early environmental enrichment moderates the behavioral and synaptic phenotype of MeCP2 null mice. Biol Psychiatry. 67, 657-665.
Luzzi S, Snowden J S, Neary D, Coccia M, Provinciali L, Lambon Ralph M A (2007) Distinct patterns of olfactory impairment in Alzheimer's disease, semantic dementia, frontotemporal dementia, and corticobasal degeneration. Neuropsychologia. 45:1823-1831.
Meusel T, Westermann B, Fuhr P, Hummel T, Welge-Lüssen A. (2010). The course of olfactory deficits in users with Parkinson's disease—a study based on psychophysical and electrophysiological measures. Neurosci Lett. 486:166-170.
Murphy C, Schubert C R, Cruickshanks K J, Klein B E, Klein R, Nondahl D M. (2002). Prevalence of olfactory impairment in older adults. JAMA. 288:2307-2312.
Nag N, Moriuchi J M, Peitzman C G, Ward B C, Kolodny N H, Berger-Sweeney J E. (2009). Environmental enrichment alters locomotor behaviour and ventricular volume in Mecp2 1lox mice. Behav Brain Res. 196, 44-48.
Nguyen A D, Shenton M E, Levitt J J. (2010). Olfactory dysfunction in schizophrenia: a review of neuroanatomy and psychophysiological measurements. Harv Rev Psychiatry. 18:279-292.
Park H L, O'Connell J E, Thomson R G. (2003). A systematic review of cognitive decline in the general elderly population. Int J Geriatr Psychiatry. 18:1121-1134.
Parrao T, Chana P, Venegas P, Behrens M I, Aylwin M L. (2012). Olfactory deficits and cognitive dysfunction in Parkinson's disease. Neurodegener Dis. 10:179-182.
Patel T R (2012). Environmental enrichment: aging and memory. Yale J Biol Med. 85:491-500.
Patel Z M, Wise S K, DelGaudio J M. (2017). Randomized controlled trial demonstrating cost-effective method of olfactory training in clinical practice: essential oils at uncontrolled concentration. Laryngoscope Invest Otolaryngol. 2:53-56.
Peters J M, Hummel T, Kratzsch T, Lötsch J, Skarke C, Frölich L. (2003). Olfactory function in mild cognitive impairment and Alzheimer's disease: an investigation using psychophysical and electrophysiological techniques. Am J Psychiatry. 160:1995-2002.
Pinto J M, Wroblewski K E, Kern D W, Schumm L P, McClintock M K. (2015). The rate of age-related olfactory decline among the general population of older U.S. adults. J Gerontol A, Biol Sci Med Sci. 70:1435-1441.
Pinto J M, Wroblewski K E, Kern D W, Schumm L P, McClintock M K (2014). Olfactory dysfunction predicts 5-year mortality in older adults. PLOS ONE. 9: e107541.
Polito L, Chierchia A, Tunesi M, Bouybayoune I, Kehoe P G, Albani D, Forloni G. (2014). Environmental enrichment lessens cognitive decline in app23 mice without affecting brain sirtuin expression. J Alzheimer's Dis. 42:851-864.
Ponsen M M, Stoffers D, Booij J, van Eck-Smit B L, Wolters E C, Berendse H W. (2004). Idiopathic hyposmia as a preclinical sign of Parkinson's disease. Ann Neurol. 56:173-181.
Restivo L, Ferrari F, Passino E, Sgobio C, Bock J, Oostra B A., Bagni C, Ammassari-Teule M. (2005). Enriched environment promotes behavioral and morphological recovery in a mouse model for the fragile X syndrome. Proc Nat Acad Sci. USA. 102:11557-11562.
Reynolds S, Urruela M, Devine D P. (2013). Effects of environmental enrichment on repetitive behaviors in the BTBR T_tf/J mouse model of autism. Autism Res. 6, 337-343.
Roberts R O, Christianson T, J H, Kremers W K, Mielke M M, Machulda M M, Vassilaki M, Alhurani R E, Geda Y E, Knopman D S, Petersen R C. (2016). Association between olfactory dysfunction and amnestic mild cognitive impairment and Alzheimer disease dementia. JAMA Neurol. 73:93-101.
Rodakowski J, Saghafi E, Butters M A, Skidmore E R. (2015). Non-pharmacological Interventions for Adults with Mild Cognitive Impairment and Early Stage Dementia: An Updated Scoping Review. Mol Aspects Med. 0, 38-53.
Ross G W, Abbott R D, Petrovitch H, Tanner C M, Davis D G, Nelson J, Markesbery W R, Hardman J, Masaki K, Launer L, White L R. (2006). Association of olfactory dysfunction with incidental Lewy bodies. Mov Disord. 21:2062-2067.
Rozenkrantz L, Zachor D, Heller I, Plotkin A, Weissbrod A, Snitz K, Secundo L, Sobel N. (2015). A mechanistic link between olfaction and autism spectrum disorder. Curr Biol. 25:1904-1910.
Sakamoto Y, Ebihara S, Ebihara T, Tomita N, Toba K, Freeman S, Arai H, Kohzuki M. (2012). Fall prevention using olfactory stimulation with lavender odor in elderly nursing home residents, a randomized controlled trial. J Am Geriatr Soc. 60:1005-1011.
Salthouse T A. (2009). When does age-related cognitive decline begin? Neurobiol Aging. 30:507-514.
Schneider T, Turczak, J, Przewlocki R. (2006). Environmental enrichment reverses behavioral alterations in rats prenatally exposed to valproic acid: Issues for a therapeutic approach in autism. Neuropsychopharmacology. 31, 36-46.
Schubert C R, Carmichael L L, Murphy C, Klein B E K, Klein R, Cruickshanks K J. (2008). Olfaction and the 5-year incidence of cognitive impairment in an epidemiologic study of older adults. J Am Geriatrics Soc. 56:1517-1521.
Schubert C R, Fischer M E, Pinto A A, Klein B E K, Klein R, Cruickshanks K J. (2017). Odor detection thresholds in a population of older adults. Laryngoscope. 127:1257-1262.
Segovia G, Yague A G, Garcia-Verdugo J M, Mora F. (2006). Environmental enrichment promotes neurogenesis and changes the extracellular concentrations of glutamate and GABA in the hippocampus of aged rats. Brain Res Bull. 70:8-14.
Segura B, Baggio H C, Solanaa, E, Palacios E M, Vendrell P, Bargalló N, Junquéa C. (2013). Neuroanatomical correlates of olfactory loss in normal aged subjects. Behav Brain Res. 246:148-153.
Seubert J J, Laukka E J, Rizzuto D, Hummel T, Fratiglioni L, Bäckman L, Larsson M. (2017). Prevalence and correlates of olfactory dysfunction in old age: a population-based study. J Gerontol A Biol Sci Med Sci. 72:1072-1079.
Sohrabi H R, Bates K A, Weinborn M G, Johnston A N B, Bahramian A, Taddei, K, Laws S M, Rodrigues M, Morici M, Howard M, Martins G, Mackay-Sim A, Gandy S E, Martins R N. (2012). Olfactory discrimination predicts cognitive decline among community-dwelling older adults. Transl Psychiatry. 2, e118-.
Swan G E, Carmelli D. (2002). Impaired olfaction predicts cognitive decline in nondemented older adults. Neuroepidemiology. 21:58-67.
Tabaton M, Monaco S, Cordone M P, Colucci M, Giaccone G, Tagliavini F, Zanusso G. (2004). Prion deposition in olfactory biopsy of sporadic Creutzfeldt-Jakob disease. Ann Neurol. 55:294-296.
Tonacci A, Billeci L, Tartarisco G, Ruta L, Muratori F, Pioggia G, Gangemi S. (2017). Olfaction in autism spectrum disorders: A systematic review. Child Neuropsychol. 23:1-25.
Toussaint N, de Roon M, van Campen J P, Kremer S, Boesveldt S. (2015). Loss of olfactory function and nutritional status in vital older adults and geriatric users. Chem Senses. 40:197-203.
Valero J, España J, Parra-Damas A, Martín E, Rodríguez-Álvarez J, Saura C A (2011). Short-term environmental enrichment rescues adult neurogenesis and memory deficits in app (sw,ind) transgenic mice. PLOS ONE. 6: e16832.
Wattendorf E, Welge-Lüssen A, Fiedler K, Bilecen D, Wolfensberger M, Fuhr P, Hummel T, Westermann B. (2009). Olfactory impairment predicts brain atrophy in Parkinson's disease. J Neurosci. 29:15410-15413.
Wegener B A, Croy I, Hähner A, Hummel T. (2018). Olfactory training with older people. Int J Geriatr Psychiatry. 33:212-220.
Williams K, Kemper S. (2010). Exploring interventions to reduce cognitive decline in aging. J Psychosoc Nurs Ment Health Serv. 48:42-51.
Williams N H, Hendry M, France B, Lewis R, Wilkinson C. (2007). Effectiveness of exercise-referral schemes to promote physical activity in adults: systematic review. Brit J Gen Practice. 57:979-986.
Woo C C, Donnelly J H, Steinberg-Epstein R, Leon M. (2015). Environmental enrichment as a therapy for autism: A clinical trial replication and extension. Behav Neurosci. 129:412-422.
Woo C C, Leon M. (2013). Environmental enrichment as an effective treatment for autism: A randomized controlled trial. Behav Neurosci. 127:487-497.
Yao L, Pinto J M, Yi X, Li L, Peng P, Wei Y. (2014). Gray matter volume reduction of olfactory cortices in users with idiopathic olfactory loss. Chem Senses. 39:755-760.

It is to be appreciated that the Detailed Description section, and not the Abstract section, is intended to be used to interpret the claims. The Abstract section may set forth one or more but not all exemplary embodiments of the present invention as contemplated by the inventors, and thus, is not intended to limit the present invention and the appended claims in any way.

The present invention has been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

The foregoing description of the specific embodiments reveals the general nature of the invention so that others can, by applying knowledge within the skill of the art, readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present invention should not be limited by any of the above-described exemplary embodiments, but should be defined only in accordance with the following claims and their equivalents.

1. A method of improving cognitive function of a user comprising:
establishing a treatment schedule including a plurality of intervals separated by breaks; and
inputting the treatment schedule into a scent-delivery device such that the device delivers one or more of a plurality of scents during each interval, and stops the delivery of the scent at the end of the interval, the plurality of scents comprising at least one scent identified in a profile of said user where the at least one identified scent is familiar to the user from a past experience.

2. The method of claim 1, wherein scents delivered during consecutive intervals are sufficiently distinct from each other so as to be distinguishable by said user.

3. The method of claim 1, wherein no scent is repeated more than once during the treatment schedule.

4. The method of claim 1, further comprising:
repeating the treatment schedule for a predetermined treatment length to improve far transfer effects for the user.

5. The method of claim 1, further comprising:
creation of the profile,
wherein creation of the profile comprises:
posing questions to the user, the questions relating to one or more of the ethnicity of the user, locations where the user has lived previously, and activities in which the user has been previously engaged;
receiving responses from the user;
based on the received responses, identifying scents corresponding to the responses; and
including, in the profile, information specifying the identified scents.

6. The method of claim 5, wherein the identifying of the scents corresponding to the responses comprises:
accessing one or more databases that relate identifiers of scents to potential responses to the posed questions; and
reading any identifiers of scents that correspond to the received responses.

7. The method of claim 1 wherein establishing the treatment schedule including the plurality of intervals separated by breaks comprises scheduling at least three intervals separated by breaks.

8. The method of claim 1 wherein inputting the treatment schedule into a scent-delivery device comprises wirelessly connecting the device to a handheld electronic device having an application that allows the treatment schedule to be uploaded from the handheld electronic device to the scent-delivery device.

9. The method of claim 1 wherein establishing a treatment schedule comprises basing an initiation of the treatment schedule on an activity signifying that a user is going to bed.

10. The method of claim 9, wherein said activity comprises communicatively coupling a handheld device to the scent-delivery device.

11. The method of claim 9, wherein said activity comprises wirelessly coupling a biometric sensor to the hand-held device, providing data that indicates the user has begun a sleep cycle.

12. A device for providing cognitive-improvement olfactory therapy comprising:
a housing defining at least one cavity, each of said at least one cavity sized to receive a removable scent cartridge, and a vent;
at least one fan adjacent the at least one cavity and capable of moving air through said at least one cavity;
at least one motor configured to activate the at least one fan;
a control board that controls the at least one motor according to a treatment schedule that includes a plurality of intervals separated by breaks, and initiates the treatment schedule at a start event;
wherein during an interval, the at least one fan is activated such that air is moved through the vent and an associated at least one cavity, thereby emitting a scent when a scent cartridge is present in the at least one cavity;
wherein the scent is one of a plurality of scents comprising at least one scent identified in a profile of a user where the at least one identified scent is familiar to the user from a past experience; and
wherein during a break, the at least one fan is inactive.

13. The device of claim 12, wherein the start event comprises a time of day.

14. The device of claim 12, wherein the start event comprises data received from a biometric sensor indicating that a user has begun a sleep cycle.

15. The device of claim 12, further comprising a personal electronic device wirelessly connected to said control board, wherein said personal electronic device provides said treatment schedule to said control board.

16. A method of improving cognitive function of a user comprising:
providing a plurality of scents categorized into scent families, each family including scent sub-groups, according to a fragrance wheel arranged such that adjacent sub-groups have similarities, the plurality of scents comprising at least one scent identified in a profile of said user where the at least one identified scent is familiar to the user from a past experience; and
establishing a treatment schedule including exposure of the user to a plurality of intervals separated by breaks, wherein a scent is administered to the user during an interval and no scent is administered during a break,
wherein scents delivered during consecutive intervals are sufficiently distinct from each other so as to be distinguishable by said user.

17. The method of claim 16, wherein no scent is repeated more than once during the treatment schedule.

18. The method of claim 16, further comprising:
repeating the treatment schedule each day for a predetermined treatment length to improve far transfer effects for the user.

19. The method of claim 16, further comprising creation of the profile, wherein the creation of the profile comprises:
posing questions to the user, the questions relating to one or more of the ethnicity of the user, locations where the user has lived previously, and activities in which the user has been previously engaged;
receiving responses from the user;
based on the received responses, identifying scents corresponding to the responses;
including, in the profile, information specifying the identified scents.

20. The method of claim 19, wherein the identifying of the scents corresponding to the responses comprises:
accessing one or more databases that map identifiers of scents to potential responses to the posed questions; and
reading any identifiers of scents that correspond to the received responses.

21. The method of claim 16, wherein administering the schedule to the user comprises using a device that automatically emits said scents according to said schedule.

22. The method of claim 21, wherein said using of the device comprises programming the device to begin the treatment schedule a predetermined amount of time after the user has gone to bed.

23. The method of claim 16, wherein the scents are presented to the user according to the treatment schedule while the user is sleeping.

24. A method for scent dispersion, the method comprising:
dispersing a first scent from a first scent container for a first duration, the first scent having a first characteristic;
providing a first dispersion break period after the first duration, wherein the first duration and the first dispersion break are based on the first characteristic; and
dispersing a second scent from a second scent container for a second duration, the second scent having a second characteristic, wherein the second duration is based on the second characteristic.

25. The scent dispersion method of claim 24, further comprises providing a second dispersion break period after the second duration, wherein the second dispersion break period is based on the second characteristic.

26. The scent dispersion method of claim 24, wherein the first or second characteristics comprise notes of the respective first or second scents.

27. The scent dispersion method of claim 26, wherein the first duration of a top note scent is longer than a duration of a scent with a middle or base note.

28. The scent dispersion method of claim 27, wherein the first dispersion break period after the first duration of the top note scent is shorter than the second dispersion break period for the second duration of a scent with middle or base note.

29. The scent dispersion method of claim 26, wherein the first duration of a middle note scent is longer than a duration of a scent with a base note.

30. The scent dispersion method of claim 29, wherein the first dispersion break period after the first duration of the middle note scent is shorter than the second break period for the second duration of the scent with the base note.
